(19) United States
(12) Patent Application Publication
(10) Pub. No.: US 2026/0274836 A1
(43) Pub. Date: Sep. 17, 2026

(54) NOVEL HEPATOSELECTIVE POLYADENYLATING POLYMERASES INHIBITORS AND THEIR METHOD OF USE

(71) Applicants: Baruch S. Blumberg Institute, Doylestown, PA (US); The University of North Carolina at Chapel Hill, Chapel Hill, NC (US)

(72) Inventors: Yanming DU, Cheshire, CT (US); Timothy M. BLOCK, Doylestown, PA (US); Nicky HWANG, Doylestown, PA (US); Stanley M. LEMON, Chapel Hill, NC (US); You LI, Cary, NC (US)

(73) Assignees: Baruch S. Blumberg Institute, Doylestown, PA (US); The University of North Carolina at Chapel Hill, Chapel Hill, NC (US)

(21) Appl. No.: 18/869,126

(22) PCT Filed: May 25, 2023
(86) PCT No.: PCT/US2023/023563

§ 371 Date: Nov. 25, 2024

Related U.S. Application Data
(60) Provisional application No. 63/345,780, filed on May 25, 2022.

(51) Int. Cl. C07D 471/04; A61K 31/4375; A61P 31/14

(52) U.S. Cl. CPC C07D 471/04; A61K 31/4375; A61P 31/14

(57) ABSTRACT

Described herein are methods of treating and preventing disease due to infection with Hepatitis A Virus in a subject in need thereof, comprising administering any one of the hepatoselective inhibitors of PAPD 5 and 7, described herein.

### CROSS-REFERENCE TO RELATED APPLICATION

This application claims the benefit of priority from U.S. Provisional Application No. 63/345,780, filed May 25, 2022; the contents of which are hereby incorporated herein by reference in their entirety.

### STATEMENT REGARDING GOVERNMENT INTERESTS

This invention was made with government support under Grant Numbers AI103083 and AI163606 awarded by the National Institutes of Health. The government has certain rights in the invention.

### FIELD OF THE DISCLOSURE

The present invention describes compounds and methods useful as hepatoselective Polyadenylating Polymerases 5 and 7 (PAPD 5 & 7) inhibitors, useful for the treatment of hepatitis A and related conditions. The present invention further describes a novel chemotype useful for the treatment of liver cancer and other diseases that involve Polyadenylating Polymerases 5 and 7 activity.

### BACKGROUND

Although the incidence of acute hepatitis A declined dramatically in the United States following the introduction of inactivated vaccines in 1995, widespread community outbreaks in recent years have led to an unprecedented resurgence of the virus with over 23,000 hospitalizations and more than 400 deaths since 2016. No effective treatment exists for the liver injury caused by hepatitis A virus (HAV), an atypical hepatotropic picornavirus that spreads by fecal-oral transmission. Other than orthotopic liver transplantation, which may be life-saving in patients with fulminant hepatitis A, there have been no advances in the treatment of this infection since discovery of the virus half a century ago. HAV is distinct from other picornaviruses in its phylogeny, structure and replication strategy. It infects the liver in a stealth-like manner, releasing newly replicated virus into the gastrointestinal track through the biliary system. Replication is noncytopathic and restricted to hepatocytes. Newly replicated virus is released without lysis from cells within small extracellular vesicles resembling exosomes. These membrane-cloaked ‘quasi-enveloped’ virions are infectious and the only form of the virus detected in blood during acute hepatitis A. Acute liver injury is caused by both adaptive and innate immune responses to infection, with both B and T cell responses involved in control of the virus.

Recently, a dihydroquinolizinone (DHQ) compound, RG-7834 (DHQ-1), has been reported to reliably reduce levels of HAV RNA and inhibit HAV replication in cultured cells. Additionally, oral treatment with RG-7834 (DHQ-1) in Ifnar1^{-/-} mice lacking type I interferon receptors blocked HAV replication, reducing intrahepatic viral RNA abundance and fecal HAV shedding, and sharply reducing serum alanine aminotransferase activities, hepatocyte apoptosis, and intrahepatic inflammatory cell infiltrates in mice with acute hepatitis A. RG7834 also completely prevented infection when given shortly after intravenous virus challenge in these mice.

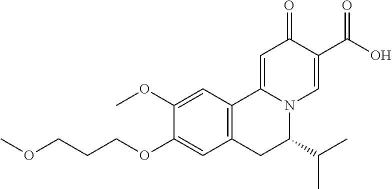 [SMILES: COCCCOc1cc2c(cc1OC)-c1cc(=O)c(C(=O)O)cn1[C@H](C(C)C)C2]

DHQ-1 is now known to be an inhibitor of the cellular (viral host) Polyadenylating Polymerases 5 and 7 (PAPD 5 & 7) encoded by the TENT4B and TENT4A genes, respectively. PAPD5 and 7 are noncanonical polyadenylating polymerases that have terminal nucleotidyltransferase activity and that mediate short 3′ polyadenylation, thereby providing a signal for the degradation of aberrant cellular RNA transcripts and maturation of a subset of non-coding transcripts. We and others have found that PAPD5 and PAPD7 inhibition is the basis of the anti-HAV activity of DHQ-1. We discovered that DHQ-1 prevents the interaction of PAPD5 with ZCCHC14, a host RNA-binding protein that binds specifically to the 5′ untranslated segment of the HAV RNA genome to which it recruits PAPD5 and PAPD7. By inhibiting the interaction of PAPD5 and PAPD7 with ZCCHC14, DHQ-1 thus blocks the recruitment of PAPD5 and PAPD7 to the HAV genome. This results in the inhibition of nascent HAV RNA synthesis, causing a reduction in HAV RNA levels. This mechanism of action is not associated with changes in the length of the 3′ poly(A) sequence of HAV RNA, and thus differs fundamentally from the antiviral mechanism by which DHQ-1 inhibits hepatitis B virus infection. This provides a novel opportunity for antiviral drug development targeting hepatitis A.

Attracted to this novel chemotype and unique mechanism, several companies and institutions have generated patent applications based on the DHQ-1 structure published in the last six years since the initial reports of DHQ-1. However, development of DHQ-1 has been frustrated because of toxicity concerns, especially, the acute neurotoxicity liabilities observed from DHQ-1. Since PAPD5 and PAPD7 are cell enzymes involved in synthesis and decay of host RNA, an effect upon host functions from systemic use of a PAPD5/7 inhibitor is not surprising. Thus, having drugs that are more selective for liver hepatocytes, which are the cells targeted by HAV, is one way to minimize or eliminate unnecessary side effects resulting from inappropriate distribution of the DHQ to other tissues. Therefore, hepatoselective PAPD5/7 inhibitors have the potential to improve the safety of PAPD5/7 inhibitors used as anti-HAV therapies.

### SUMMARY

The present invention is directed toward novel hepatoselective inhibitors of PAPD5 and PAPD7, compounds of formula (I):

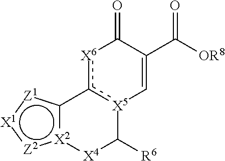

Including hydrates, solvates, pharmaceutically acceptable salts, isotopic isomers, prodrugs and complexes thereof, wherein:
X¹ is selected from the group consisting of

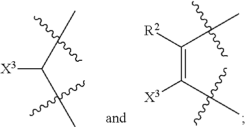

When X¹ is

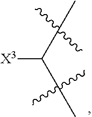

X² is nitrogen;
When X¹ is

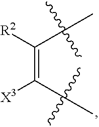

X² is carbon;
Z¹ is selected from the group consisting of N and CR¹;
Z² is selected from the group consisting of N and CR⁴;
Z¹ and Z² are not N at the same time;
X³ is selected from the group consisting of CH₂R, —OR, —NR₃R and

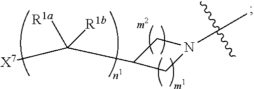

X⁴ is selected from the group consisting of CHR⁵(CH₂)ₚ, O(CH₂)ₚ, S(CH₂)ₚ, SO(CH₂)ₚ, and SO₂(CH₂)ₚ, and p is 0, 1, or 2;
X⁵ is selected from the group consisting of N and C;
X⁶ is selected from the group consisting of CR⁷ and NR⁷;
When X⁵ is N, X⁶ is CR⁷;
When X⁵ is C, X⁶ is NR⁷;
When X⁵ is N, X⁴ is CHR⁵(CH₂)ₚ, S(CH₂)ₚ, SO(CH₂)ₚ, and SO₂(CH₂)ₚ, p is 0, 1, or 2;
When X⁵ is N, X⁴ is O(CH₂)ₚ, p is 0, 1, or 2;
When X⁵ is C, X⁴ is selected from the group consisting of O(CH₂)ₚ, S(CH₂)ₚ, SO(CH₂)ₚ, and SO₂(CH₂)ₚ, p is 0, 1, or 2;
R is selected from the group consisting of

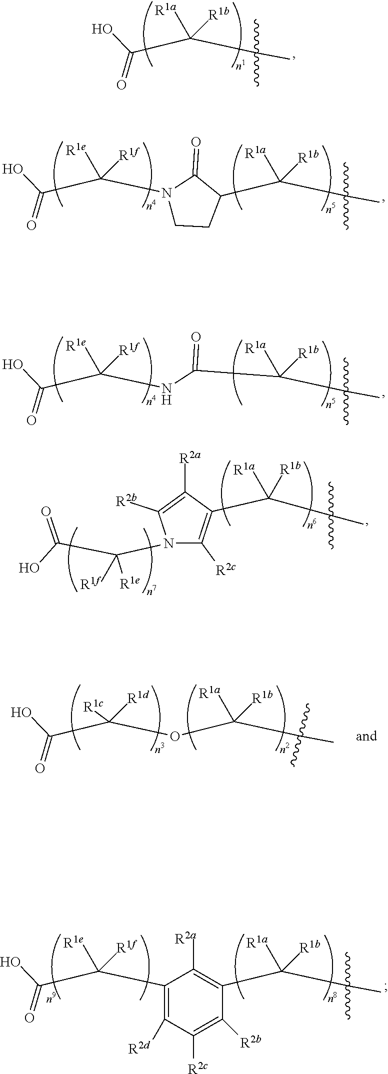

n¹ is 1, 2, 3, 4, 5, 6, 7, 8, 9, 10, 11, 12, 13, 14, 15, or 16;
n² is 2, 3, 4, 5, 6, 7, or 8;
n³ is 1, 2, 3, 4, 5, 6, 7, or 8;
n⁴ is 1, 2, 3, 4, 5, 6, or 7;
n⁵ is 1, 2, 3, 4, 5, 6, or 7;
n⁶ is 1, 2, 3, 4, 5, 6, or 7;
n⁷ is 1, 2, 3, 4, 5, 6, or 7;
n⁸ is 1, 2, 3, 4, 5, 6, or 7;
n⁹ is 0, 1, 2, 3, 4, 5, 6, or 7;
   The sum of n² and n³ does not exceed 14:
   The sum of n⁴ and n⁵ does not exceed 14:
   The sum of n⁶ and n⁷ does not exceed 14:
   The sum of n⁸ and n⁹ does not exceed 14:
m¹ is 1, 2 or 3;
m² is 0, 1, 2, or 3;
R¹ is selected from the group consisting of H, halogen, OH, CN, C₁₋₆ alkyl, C₁₋₆ haloalkyl, C₃₋₇ branched alkyl, and C₃₋₇ cycloalkyl;
R^{1a} is at each occurrence independently selected from the group hydrogen, halogen, C₁₋₆ alkyl, C₃₋₇ branched alkyl, and C₃₋₇ cycloalkyl;
R^{1b} is at each occurrence independently selected from the group hydrogen, halogen, C₁₋₆ alkyl, C₃₋₇ branched alkyl, and C₃₋₇ cycloalkyl;
R^{1c} is at each occurrence independently selected from the group hydrogen, halogen, C₁₋₆ alkyl, C₃₋₇ branched alkyl, and C₃₋₇ cycloalkyl;
R^{1d} is at each occurrence independently selected from the group hydrogen, halogen, C₁₋₆ alkyl, C₃₋₇ branched alkyl, and C₃₋₇ cycloalkyl;
R^{1e} is at each occurrence independently selected from the group hydrogen, halogen, C₁₋₆ alkyl, C₃₋₇ branched alkyl, and C₃₋₇ cycloalkyl;
R^{1f} is at each occurrence independently selected from the group hydrogen, halogen, C₁₋₆ alkyl, C₃₋₇ branched alkyl, and C₃₋₇ cycloalkyl;
R² is selected from the group consisting of H, halogen, C₁₋₆ alkyl, C₃₋₇ cycloalkyl, C₁₋₆ haloalkyl, C₃₋₇ branched alkyl, C₁₋₆ alkoxy, C₁₋₆ haloalkoxy, C₃₋₇ branched alkoxy, CN, and aromatic ring with 0-3 heteroatoms;
In some embodiments R¹ and R² are taken together with the atoms to which they are bound to form a 5-7 membered ring that contains 0-2 heteroatoms;
R³ is selected from group consisting of hydrogen, C₁₋₆ alkyl, and C₂₋₆ alkene;
R⁴ is selected from the group consisting of H, halogen, C₁₋₆ alkyl, C₁₋₆ alkoxy, and CN;
In some embodiments R³ and R⁴ are taken together to with the atoms to which they are bound to form a 5-7 membered ring with 0-2 heteroatoms;
In some embodiments R³ and R⁴ are taken together to with the atoms to which they are bound to form a 5-6 membered aromatic ring with 0-2 heteroatoms;
R⁵ is selected from the group consisting of H, C₁₋₆ alkyl, and C₃₋₇ branch alkyl;
R⁶ is selected from the group consisting of H, C₁₋₁₀ alkyl, C₁₋₁₀ haloalkyl, C₃₋₇ branched alkyl, C₃₋₇cycloalkyl, and optionally substituted aryl;
In some embodiments R⁵ and R⁶ are taken together to with the atoms to which they are bound to form a 5-7 membered ring;
When X⁶ is CR⁷, R⁷ is selected from the group consisting of H, halogen, C₁₋₆ alkyl, C₃₋₇cycloalkyl, C₃₋₇ branched alkyl, and CN;
When X⁶ is NR⁷, R⁷ is selected from the group consisting of H, C₁₋₆ alkyl, C₃₋₇ cycloalkyl, and C₃₋₇ branched alkyl;
R⁸ is selected from the group consisting of is H and C₁₋₆ alkyl.

The present invention further relates to compositions comprising: an effective amount of one or more compounds according to the present invention and an excipient.

The present invention also relates to a method for treating or preventing diseases that involve PAPD5 and PAPD7, including, for example, hepatitis A, said method comprising administering to a subject an effective amount of a compound or composition according to the present invention.

The present invention yet further relates to a method for treating or preventing diseases that involve PAPD5 and PAPD7, including, for example, hepatitis A, wherein said method comprises administering to a subject a composition comprising an effective amount of one or more compounds according to the present invention and an excipient.

The present invention also relates to a method for treating or preventing disease or conditions associated with hepatitis A, and diseases that involve PAPD5 and PAPD7. Said methods comprise administering to a subject an effective amount of a compound or composition according to the present invention.

The present invention yet further relates to a method for treating or preventing disease or conditions associated with hepatitis A, and diseases that involve PAPD5 and PAPD7, wherein said method comprises administering to a subject a composition comprising an effective amount of one or more compounds according to the present invention and an excipient.

The present invention also relates to a method for treating or preventing disease or conditions associated with PAPD5 and PAPD7. Said methods comprise administering to a subject an effective amount of a compound or composition according to the present invention.

The present invention yet further relates to a method for treating or preventing disease or conditions associated with PAPD5 and PAPD7, wherein said method comprises administering to a subject a composition comprising an effective amount of one or more compounds according to the present invention and an excipient.

The present invention further relates to a process for preparing the hepatoselective PAPD5 and PAPD7 inhibitors of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### BRIEF DESCRIPTION OF THE DRAWINGS

FIG. 1 depicts HAV replication inhibition by 65042-E-OH (see, Example 6) and 65075 (see, Example 8)

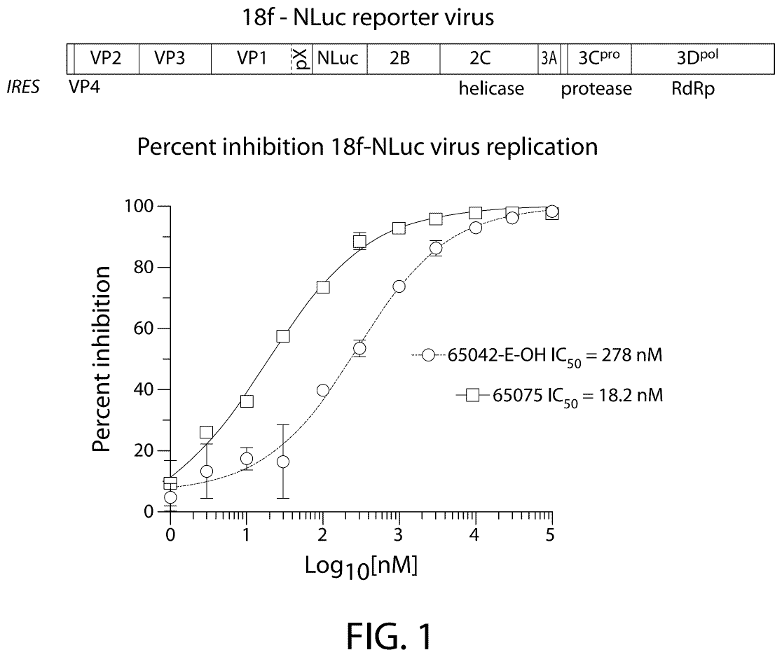

FIG. 2 depicts the results of cell toxicity studies involving certain compounds described herein using the WST-1 Cell Proliferation Reagent for 65042-E-OH (see, Example 6) and 65075 (see, Example 8).

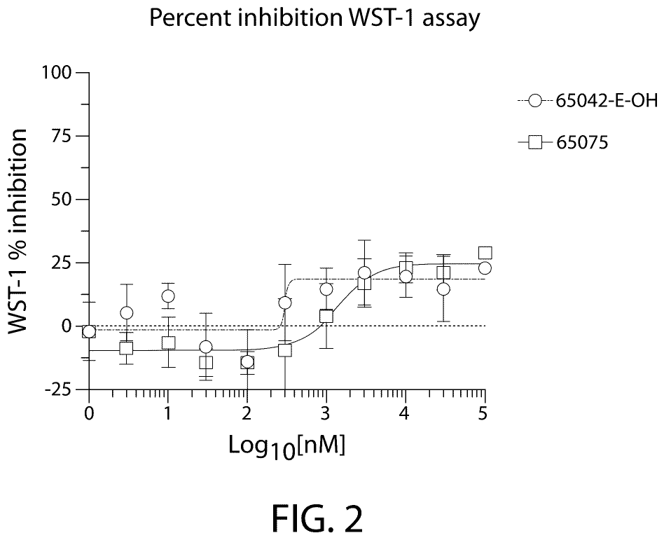

### DETAILED DESCRIPTION

The hepatoselective inhibitors of PAPD 5 and 7 agents of the present invention are capable of treating and preventing diseases associated with hepatitis A virus. It has been discovered that compounds of the disclosure are inhibitors of PAPD 5 and 7. In addition, it has been determined that compounds of the disclosure are substrates for organic anion transporting poly-peptide protein 1B1 (OATP1B1) and organic anion transporting poly-peptide protein 1B3 (OATP1B3), a feature not seen in related compounds such as DHQ-1. It has further been determined that the ability of compounds of the disclosure to act as substrates of a OATP1B1 and OATP1B3 leads to unexpected, preferential concentration of compound in the liver in comparison to the systemic circulation.

As used herein, the terms “hepatoselective”, “liver selective” and “hepatotropic” may be used interchangeably.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆ alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

“Haloalkoxy” refers to the group —O-alkyl, wherein the alkyl group is as defined above wherein the —O-alkyl is substituted with 1 or more halogen. Haloalkoxy groups include perhaloalkyl groups, wherein all hydrogens of an alkoxy group have been replaced with halogens (e.g., —OCF₃, —OCF₂CF₃). Haloalkoxy groups can optionally be substituted with one or more substituents in addition to halogen. Examples of haloalkoxy groups include, but are not limited to, fluoromethoxy, dichloroethoxy, trifluoromethoxy, trichloromethoxy, pentafluoroethoxy, and pentachloroethoxy groups.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

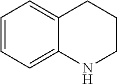

is, for the purposes of the present invention, considered a heterocyclic unit. 6,7-Dihydro-5H-cyclopentapyrimidine having the formula:

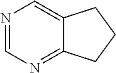

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

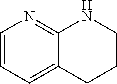

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine (I)), —CN, —NO₂, oxo (═O), —OR⁹, —SR⁹, —N(R⁹)₂, —NR⁹C(O)R⁹, —SO₂R⁹, —SO₂OR⁹, —SO₂N(R⁹)₂, —C(O)R⁹, —C(O)OR⁹, —C(O)N(R⁹)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R⁹; wherein R⁹, at each occurrence, independently is hydrogen, —OR¹⁰, —SR¹⁰, —C(O)R¹⁰, —C(O)OR¹⁰, —C(O)N(R¹⁰)₂, —SO₂R¹⁰, —S(O)₂OR¹⁰, —N(R¹⁰)₂, —NR¹⁰C(O)R¹⁰, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁹ units taken together with the atom(s) to which they are bound form an optionally substituted carbocycle or heterocycle wherein said carbocycle or heterocycle has 3 to 7 ring atoms; wherein R¹⁰, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹⁰ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹¹; for example, —OH, —OCH₃, —OCH₂CH₃, —OCH₂CH₂CH₃;
ii) —C(O)R¹¹; for example, —COCH₃, —COCH₂CH₃, —COCH₂CH₂CH₃;
iii) —C(O)OR¹¹; for example, —CO₂CH₃, —CO₂CH₂CH₃, —CO₂CH₂CH₂CH₃;
iv) —C(O)N(R¹)₂; for example, —CONH₂, —CONHCH₃, —CON(CH₃)₂;
v) —N(R¹)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹¹; for example, —SO₂H; —SO₂CH₃; —SO₂C₆H₅;
ix) C₁-C₆ linear, branched, or cyclic alkyl;
x) Cyano
xi) Nitro;
xii) N(R¹¹)C(O)R¹¹;
xiii) Oxo (═O);
xiv) Heterocycle; and
xv) Heteroaryl.
   wherein each R¹¹ is independently hydrogen, optionally substituted C₁-C₆ linear or branched alkyl (e.g., optionally substituted C₁-C₄ linear or branched alkyl), or optionally substituted C₃-C₆cycloalkyl (e.g optionally substituted C₃-C₄ cycloalkyl); or two R¹¹ units can be taken together to form a ring comprising 3-7 ring atoms. In certain aspects, each R¹¹ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁—C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the hepatoselective Polyadenylating Polymerases 5 and 7 (PAPD 5 & 7) inhibitors described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R¹⁰)₂, each R¹⁰ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

### The Hepatoselective Polyadenylating Polymerases 5 and 7 Inhibitors

The hepatoselective Polyadenylating Polymerases 5 and 7 inhibitors of the present invention are functionalized pyridin-4(1H)-ones, and include all enantiomeric and diastereomeric forms and pharmaceutically accepted salts thereof having the formula (I):

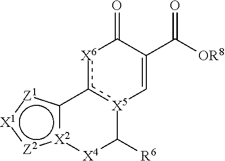

Including hydrates, solvates, pharmaceutically acceptable salts, prodrugs and complexes thereof, wherein:
X¹ is selected from the group consisting of

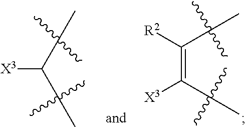

When X¹ is

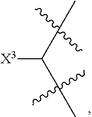

X² is nitrogen;
When X¹ is

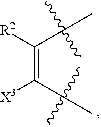

X² is carbon;
Z¹ is selected from the group consisting of N and CR¹;
Z² is selected from the group consisting of N and CR⁴;
Z¹ and Z² are not N at the same time;
X³ is selected from the group consisting of CH₂R, —OR, —NR₃R an

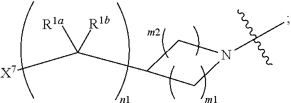

X⁴ is selected from the group consisting of CHR⁵ (CH₂)ₚ, O(CH₂)ₚ, S(CH₂)ₚ, SO(CH₂)ₚ, and SO₂(CH₂)ₚ, p is 0, 1, or 2;
X⁵ is selected from the group consisting of N and C;
X⁶ is selected from the group consisting of CR⁷ and NR⁷;
When X⁵ is N, X⁶ us CR⁷;
When X⁵ is C, X⁶ is NR⁷;
When X⁵ is N, X⁴ is CHR⁵(CH₂)ₚ, p is 0, 1, or 2;
When X⁵ is N, X⁴ is O(CH₂)ₚ, S(CH₂)ₚ, SO(CH₂)ₚ, and SO₂(CH₂)ₚ, p is 0, 1, or 2.
When X⁵ is C, X⁴ is selected from the group consisting of O(CH₂)ₚ, S(CH₂)ₚ, SO(CH₂)ₚ, and SO₂(CH₂)ₚ, p is 0, 1, or 2;
R is selected from the group consisting of

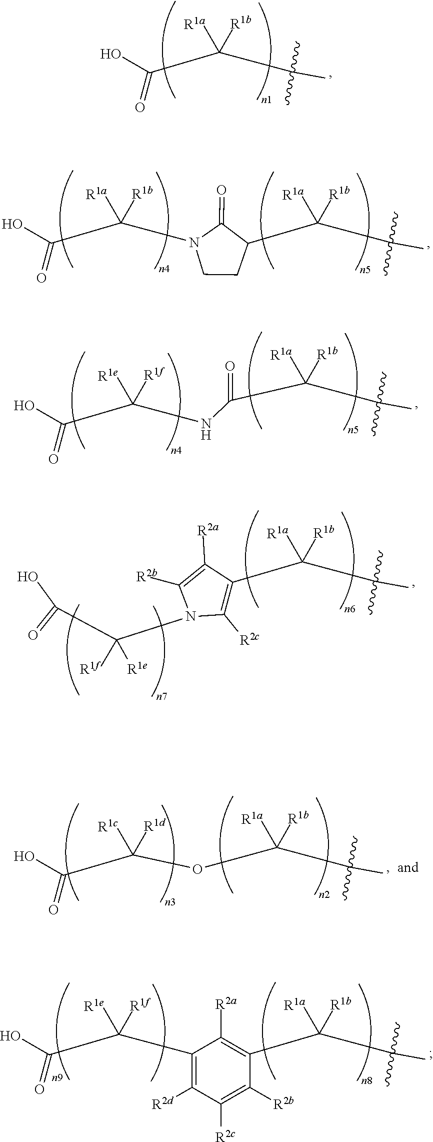

n¹ is 1, 2, 3, 4, 5, 6, 7, 8, 9, 10, 11, 12, 13, 14, 15, or 16;
n² is 2, 3, 4, 5, 6, 7, or 8;
n³ is 1, 2, 3, 4, 5, 6, 7, or 8;
n⁴ is 1, 2, 3, 4, 5, 6, or 7;
n⁵ is 1, 2, 3, 4, 5, 6, or 7;
n⁶ is 1, 2, 3, 4, 5, 6, or 7;
n⁷ is 1, 2, 3, 4, 5, 6, or 7;
n⁸ is 1, 2, 3, 4, 5, 6, or 7;
n⁹ is 0, 1, 2, 3, 4, 5, 6, or 7;
The sum of n² and n³ does not exceed 14:
The sum of n⁴ and n⁵ does not exceed 14:
The sum of n⁶ and n⁷ does not exceed 14:
The sum of n⁸ and n⁹ does not exceed 14:
m¹ is 1, 2, or 3;
m² is 0, 1, 2, or 3;
R¹ is selected from the group consisting of H, halogen, OH, CN, C₁₋₆ alkyl, C₁₋₆ haloalkyl, C₃₋₇ branched alkyl, and C₃₋₇ cycloalkyl;
R^{1a} is at each occurrence independently selected from the group hydrogen, halogen, C₁₋₆ alkyl, C₃₋₇ branched alkyl, and C₃₋₇ cycloalkyl;
R^{1b} is at each occurrence independently selected from the group hydrogen, halogen, C₁₋₆ alkyl, C₃₋₇ branched alkyl, and C₃₋₇ cycloalkyl;
R^{1c} is at each occurrence independently selected from the group hydrogen, halogen, C₁₋₆ alkyl, C₃₋₇ branched alkyl, and C₃₋₇ cycloalkyl;
R^{1d} is at each occurrence independently selected from the group hydrogen, halogen, C₁₋₆ alkyl, C₃₋₇ branched alkyl, and C₃₋₇ cycloalkyl;
R^{1e} is at each occurrence independently selected from the group hydrogen, halogen, C₁₋₆ alkyl, C₃₋₇ branched alkyl, and C₃₋₇ cycloalkyl;
R^{1f} is at each occurrence independently selected from the group hydrogen, halogen, C₁₋₆ alkyl, C₃₋₇ branched alkyl, and C₃₋₇ cycloalkyl;
R² is selected from the group consisting of H, halogen, C₁₋₆ alkyl, C₃₋₇ cycloalkyl, C₁₋₆ haloalkyl, C₃₋₇ branched alkyl, C₁₋₆ alkoxy, C₁₋₆ haloalkoxy, C₃₋₇ branched alkoxy, CN, and aromatic ring with 0-3 heteroatoms;
In some embodiments R¹ and R² are taken together to with the atoms to which they are bound to form a 5-7 membered ring that contains 0-2 heteroatoms;
R³ is selected from group consisting of hydrogen, C₁₋₆ alkyl, and C₂₋₆ alkene;
R⁴ is selected from the group consisting of H, halogen, C₁₋₆ alkyl, C₁₋₆ alkoxy, and CN;
In some embodiments R³ and R⁴ are taken together with the atoms to which they are bound to form a 5-7 membered ring with 0-2 heteroatoms.
In some embodiments R³ and R⁴ are taken together to with the atoms to which they are bound to form a 5-6 membered aromatic ring with 0-2 heteroatoms;
R⁵ is selected from the group consisting of H, C₁₋₆ alkyl, and C₃₋₇ branch alkyl;
R⁶ is selected from the group consisting of H, C₁₋₁₀ alkyl C₁₋₁₀ haloalkyl, C₃₋₇ branched alkyl, C₃₋₇ cycloalkyl, and optionally substituted aryl.

In some embodiments R⁵ and R⁶ are taken together to with the atoms to which they are bound to form a 5-7 membered ring;
When X⁶ is CR⁷, R⁷ is selected from the group consisting of H, halogen, C₁₋₆ alkyl, C₃. 7 cycloalkyl, C₃₋₇ branched alkyl, and CN;
When X⁶ is NR⁷, R⁷ is selected from the group consisting of H, C₁₋₆ alkyl, C₃₋₇ cycloalkyl, and C₃₋₇ branched alkyl;
R⁸ is selected from the group consisting of is H and C₁₋₆ alkyl.

The compounds of the present invention include compounds having formula (II):

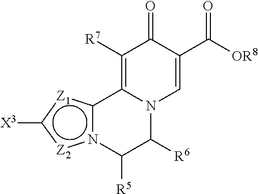

Including hydrates, solvates, pharmaceutically acceptable salts, prodrugs, isotopic isomers, and complexes thereof.

The compounds of the present invention include compounds having formula (III):

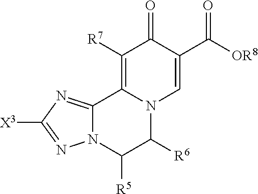

Including hydrates, solvates, pharmaceutically acceptable salts, prodrugs, isotopic isomers, and complexes thereof.

The compounds of the present invention include compounds having formula (IV):

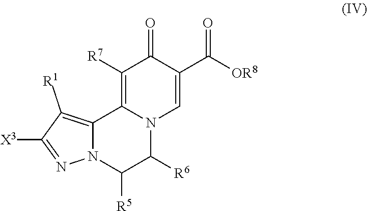

Including hydrates, solvates, pharmaceutically acceptable salts, prodrugs, isotopic isomers, and complexes thereof.

The compounds of the present invention include compounds having formula (V):

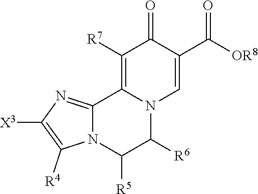

Including hydrates, solvates, pharmaceutically acceptable salts, prodrugs, isotopic isomers, and complexes thereof.

The compounds of the present invention include compounds having formula (VI):

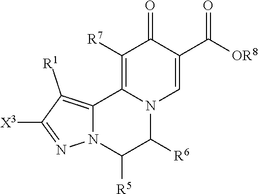

Including hydrates, solvates, pharmaceutically acceptable salts, prodrugs, isotopic isomers, and complexes thereof.

The compounds of the present invention include compounds having formula (VII):

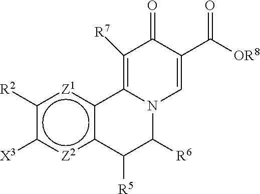

Including hydrates, solvates, pharmaceutically acceptable salts, prodrugs, isotopic isomers, and complexes thereof.

The compounds of the present invention include compounds having formula (VIII):

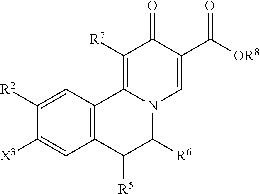

Including hydrates, solvates, pharmaceutically acceptable salts, prodrugs, isotopic isomers, and complexes thereof.

The compounds of the present invention include compounds having formula (IX):

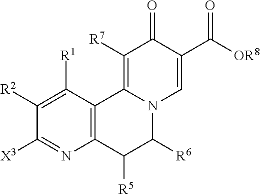

Including hydrates, solvates, pharmaceutically acceptable salts, prodrugs isotopic isomers, and complexes thereof.

The compounds of the present invention include compounds having formula (X):

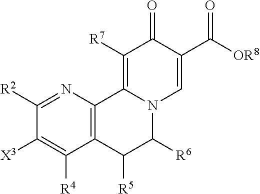

Including hydrates, solvates, pharmaceutically acceptable salts, prodrugs, isotopic isomers, and complexes thereof.

The compounds of the present invention include compounds having formula (XI):

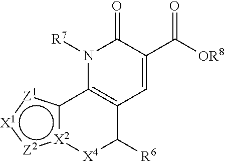

Including hydrates, solvates, pharmaceutically acceptable salts, prodrugs, isotopic isomers, and complexes thereof.

The compounds of the present invention include compounds having formula (XII):

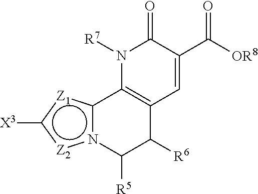

Including hydrates, solvates, pharmaceutically acceptable salts, prodrugs, isotopic isomers, and complexes thereof.

The compounds of the present invention include compounds having formula (XIII):

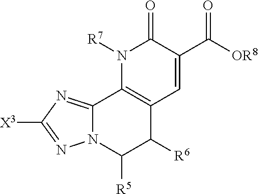

Including hydrates, solvates, pharmaceutically acceptable salts, prodrugs, isotopic isomers, and complexes thereof.

The compounds of the present invention include compounds having formula (XIV):

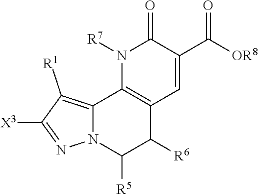

Including hydrates, solvates, pharmaceutically acceptable salts, prodrugs, isotopic isomers, and complexes thereof.

The compounds of the present invention include compounds having formula (XV):

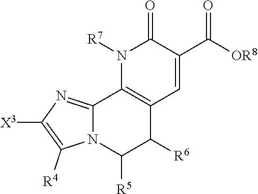

Including hydrates, solvates, pharmaceutically acceptable salts, prodrugs, isotopic isomers, and complexes thereof.

The compounds of the present invention include compounds having formula (XVI):

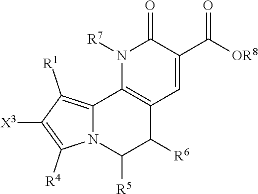

Including hydrates, solvates, pharmaceutically acceptable salts, prodrugs, isotopic isomers, and complexes thereof.

The compounds of the present invention include compounds having formula (XVII):

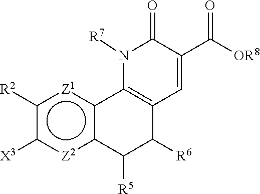

Including hydrates, solvates, pharmaceutically acceptable salts, prodrugs, isotopic isomers, and complexes thereof.

The compounds of the present invention include compounds having formula (XVIII):

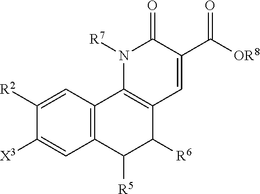

Including hydrates, solvates, pharmaceutically acceptable salts, prodrugs, isotopic isomers, and complexes thereof.

The compounds of the present invention include compounds having formula (XIX):

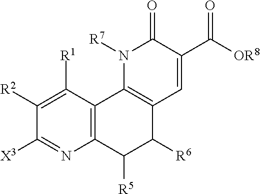

Including hydrates, solvates, pharmaceutically acceptable salts, prodrugs, isotopic isomers, and complexes thereof.

The compounds of the present invention include compounds having formula (XX):

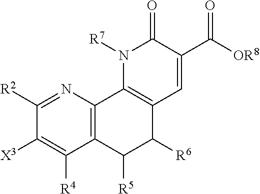

Including hydrates, solvates, pharmaceutically acceptable salts, prodrugs, isotopic isomers, and complexes thereof.

In some embodiments X¹ is

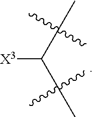

In some embodiments X¹ is

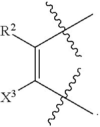

In some embodiments X² is N.

In some embodiments X² is C.

In some embodiments Z¹ is N.

In some embodiments Z¹ is CR¹.

In some embodiments Z² is N.

In some embodiments Z² is CR⁴.

In some embodiments X³ is CH₂R.

In some embodiments X³ is —OR.

In some embodiments X³ is —NR₃R.

In some embodiments X³ is

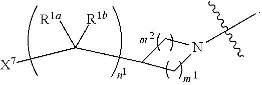

In some embodiments X⁴ is CHR⁵

In some embodiments X⁴ is, O.

In some embodiments X⁴ is S.

In some embodiments X⁴ is SO.

In some embodiments X⁴ is SO₂.

In some embodiments X⁵ is N.

In some embodiments X⁵ is C.

In some embodiments X⁶ is CR⁷.

In some embodiments X⁶ is NR⁷.

In some embodiments R is

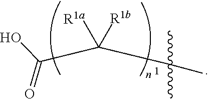

In some embodiments R is

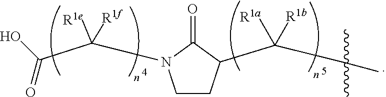

In some embodiments R is

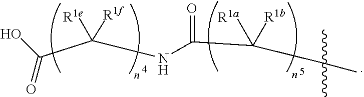

In some embodiments R is

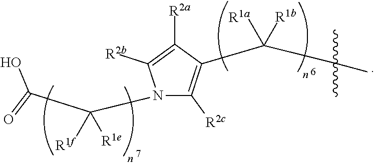

In some embodiments R is

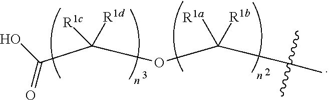

In some embodiments R is

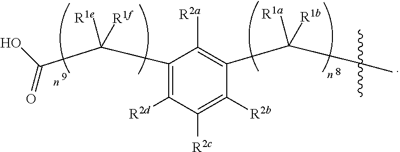

In some embodiments n¹ is 1.

In some embodiments n¹ is 2.

In some embodiments n¹ is 3.

In some embodiments n¹ is 4.

In some embodiments n¹ is 5.

In some embodiments n¹ is 6.

In some embodiments n¹ is 7.

In some embodiments n¹ is 8.

In some embodiments n¹ is 9.

In some embodiments n¹ is 10.

In some embodiments n¹ is 11.

In some embodiments n¹ is 12.

In some embodiments n¹ is 13.

In some embodiments n¹ is 14.

In some embodiments n¹ is 15.

In some embodiments n¹ is 16.

In some embodiments n² is 2.

In some embodiments n² is 3.

In some embodiments n² is 4.

In some embodiments n² is 5.

In some embodiments n² is 6.

In some embodiments n² is 7.

In some embodiments n² is 8.

In some embodiments n³ is 1.

In some embodiments n³ is 2.

In some embodiments n³ is 3.

In some embodiments n³ is 4.

In some embodiments n³ is 5.

In some embodiments n³ is 6.

In some embodiments n³ is 7.

In some embodiments n³ is 8.

In some embodiments n⁴ is 1.

In some embodiments n⁴ is 2.

In some embodiments n⁴ is 3.

In some embodiments n⁴ is 4.

In some embodiments n⁴ is 5.

In some embodiments n⁴ is 6.

In some embodiments n⁴ is 7.

In some embodiments n⁵ is 1.

In some embodiments n⁵ is 2.

In some embodiments n⁵ is 3.

In some embodiments n⁵ is 4.

In some embodiments n⁵ is 5.

In some embodiments n⁵ is 6.

In some embodiments n⁵ is 7.

In some embodiments n⁶ is 1.

In some embodiments n⁶ is 2.

In some embodiments n⁶ is 3.

In some embodiments n⁶ is 4.

In some embodiments n⁶ is 5.

In some embodiments n⁶ is 6.

In some embodiments n⁶ is 7.

In some embodiments n⁷ is 1.

In some embodiments n⁷ is 2.

In some embodiments n⁷ is 3.

In some embodiments n⁷ is 4.

In some embodiments n⁷ is 5.

In some embodiments n⁷ is 6.

In some embodiments n⁷ is 7.

In some embodiments n⁸ is 1.

In some embodiments n⁸ is 2.

In some embodiments n⁸ is 3.

In some embodiments n⁸ is 4.

In some embodiments n⁸ is 5.

In some embodiments n⁸ is 6.

In some embodiments n⁸ is 7.

In some embodiments n⁹ is 0.

In some embodiments n⁹ is 1.

In some embodiments n⁹ is 2.

In some embodiments n⁹ is 3.

In some embodiments n⁹ is 4.

In some embodiments n⁹ is 5.

In some embodiments n⁹ is 6.

In some embodiments n⁹ is 7.

In some embodiments m¹ is 1.

In some embodiments m¹ is 2.

In some embodiments m¹ is 3.

In some embodiments m² is 0.

In some embodiments m² is 1.

In some embodiments m² is 2.

In some embodiments m² is 3.

In some embodiments R¹ is H.

In some embodiments R¹ is halogen.

In some embodiments R¹ is OH.

In some embodiments R¹ is CN.

In some embodiments R¹ is C₁₋₆ alkyl.

In some embodiments R¹ is C₁₋₆ haloalkyl.

In some embodiments R¹ is C₃₋₇ branched alkyl.

In some embodiments R¹ is C₃₋₇ cycloalkyl.

In some embodiments R^{1a} is hydrogen.

In some embodiments R^{1a} is halogen.

In some embodiments R^{1a} is C₁₋₆ alkyl.

In some embodiments R^{1a} is C₃₋₇ branched alkyl

In some embodiments R^{1a} is C₃₋₇ cycloalkyl.

In some embodiments R^{1b} is hydrogen.

In some embodiments R^{1b} is halogen.

In some embodiments R^{1b} is C₁₋₆ alkyl.

In some embodiments R^{1b} is C₃₋₇ branched alkyl.

In some embodiments R^{1c} is C₃₋₇ cycloalkyl.

In some embodiments R^{1c} is hydrogen.

In some embodiments R^{1c} is halogen.

In some embodiments R^{1c} is C₁₋₆ alkyl.

In some embodiments R^{1c} is C₃₋₇ branched alkyl.

In some embodiments R^{1c} is C₃₋₇ cycloalkyl.

In some embodiments R^{1d} is hydrogen.

In some embodiments R^{1d} is halogen.

In some embodiments R^{1d} is C₁₋₆ alkyl.

In some embodiments R^{1d} is C₃₋₇ branched alkyl.

In some embodiments R^{1d} is C₃₋₇ cycloalkyl.

In some embodiments R^{1e} is hydrogen.

In some embodiments R^{1e} is halogen.

In some embodiments R^{1e} is C₁₋₆ alkyl.

In some embodiments R^{1e} is C₃₋₇ branched alkyl.

In some embodiments R^{1e} is C₃₋₇ cycloalkyl.

In some embodiments R^{1f} is hydrogen.

In some embodiments R^{1f} is halogen.

In some embodiments R^{1f} is C₁₋₆ alkyl.

In some embodiments R^{1f} is C₃₋₇ branched alkyl.

In some embodiments R^{1f} is C₃₋₇ cycloalkyl.

In some embodiments R² is H.

In some embodiments R² is halogen.

In some embodiments R² is C₁₋₆ alkyl.

In some embodiments R² is C₁₋₆ haloalkyl.

In some embodiments R² is C₃₋₇ branched alkyl.

In some embodiments R² is C₁₋₆ alkoxy.

In some embodiments R² is C₁₋₆haloalkoxy.

In some embodiments R² is C₃₋₇ branched alkoxy.

In some embodiments R² is CN.

In some embodiments R² is aromatic ring with 0-3 heteroatoms.

In some embodiments R² is C₃₋₇cycloalkyl.

In some embodiments R¹ and R² are taken together to with the atoms to which they are bound to form a 5 membered ring that contains 0-2 heteroatoms.

In some embodiments R¹ and R² are taken together to with the atoms to which they are bound to form a 6 membered ring that contains 0-2 heteroatoms.

In some embodiments R¹ and R² are taken together to with the atoms to which they are bound to form a 7 membered ring that contains 0-2 heteroatoms.

In some embodiments R³ is hydrogen.

In some embodiments R³ is C₁₋₆ alkyl.

In some embodiments R³ is C₂₋₆ alkene.

In some embodiments R⁴ is H.

In some embodiments R⁴ is halogen.

In some embodiments R⁴ is C₁₋₆ alkyl.

In some embodiments R⁴ is C₁₋₆ alkoxy.

In some embodiments R⁴ is CN.

In some embodiments R³ and R⁴ are taken together with the atoms to which they are bound to form a 5 membered ring with 0-2 heteroatoms.

In some embodiments R³ and R⁴ are taken together with the atoms to which they are bound to form a 6 membered ring with 0-2 heteroatoms.

In some embodiments R³ and R⁴ are taken together with the atoms to which they are bound to form a 7 membered ring with 0-2 heteroatoms.

In some embodiments R³ and R⁴ are taken together with the atoms to which they are bound to form a 5 membered aromatic ring with 0-2 heteroatoms.

In some embodiments R³ and R⁴ are taken together with the atoms to which they are bound to form a 6 membered aromatic ring with 0-2 heteroatoms.

In some embodiments R⁵ is H.

In some embodiments R⁵ is C₁₋₆ alkyl.

In some embodiments R⁵ is C₃₋₇ branch alkyl.

In some embodiments R⁶ is H.

In some embodiments R⁶ is C₁₋₁₀ alkyl.

In some embodiments R⁶ is C₁₋₁₀ haloalkyl.

In some embodiments R⁶ is C₃₋₇ branched alkyl.

In some embodiments R⁶ is C₃₋₇cycloalkyl.

In some embodiments R⁶ is optionally substituted aryl.

In some embodiments R⁵ and R⁶ are taken together to with the atoms to which they are bound to form a 5 membered ring.

In some embodiments R⁵ and R⁶ are taken together to with the atoms to which they are bound to form a 6 membered ring.

In some embodiments R⁵ and R⁶ are taken together to with the atoms to which they are bound to form a 7 membered ring.

In some embodiments R⁷ is H.

In some embodiments R⁷ is halogen.

In some embodiments R⁷ is C₁₋₆ alkyl.

In some embodiments R⁷ is C₃₋₇ cycloalkyl.

In some embodiments R⁷ is C₃₋₇ branch alkyl.

In some embodiments R⁷ is CN.

In some embodiments R⁸ is H.

In some embodiments R⁸ is C₁₋₆ alkyl.

For the purposes of demonstrating the manner in which the compounds of the present invention are named and referred to herein, the compound having the formula:

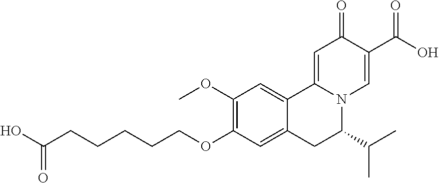 [SMILES: COc1cc2c(cc1OCCCCCC(=O)O)C[C@@H](C(C)C)n1cc(C(=O)O)c(=O)cc1-2]

has the chemical name (S)-9-((5-carboxypentyl)oxy)-6-isopropyl-10-methoxy-2-oxo-6,7-dihydro-2H-pyrido[2,1-a]isoquinoline-3-carboxylic acid.

For the purposes of demonstrating the manner in which the compounds of the present invention are named and referred to herein, the compound having the formula:

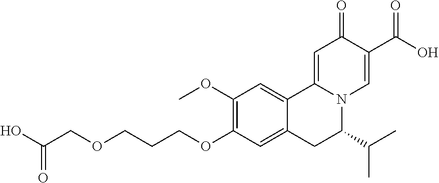 [SMILES: COc1cc2c(cc1OCCCOCC(=O)O)C[C@@H](C(C)C)n1cc(C(=O)O)c(=O)cc1-2]

has the chemical name (S)-9-(3-(carboxymethoxy)propoxy)-6-isopropyl-10-methoxy-2-oxo-6,7-dihydro-2H-pyrido[2,1-a]isoquinoline-3-carboxylic acid.

For the purposes of the present invention, a compound depicted by the racemic formula, for example:

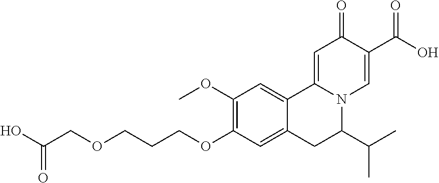 [SMILES: COc1cc2c(cc1OCCCOCC(=O)O)CC(C(C)C)n1cc(C(=O)O)c(=O)cc1-2]

will stand equally well for either of the two enantiomers having the formula:

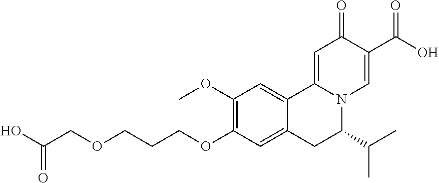 [SMILES: COc1cc2c(cc1OCCCOCC(=O)O)C[C@@H](C(C)C)n1cc(C(=O)O)c(=O)cc1-2]

or the formula:

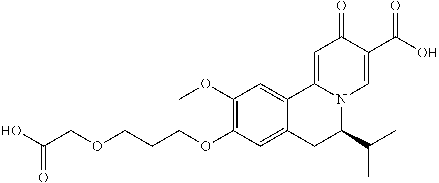 [SMILES: COc1cc2c(cc1OCCCOCC(=O)O)C[C@H](C(C)C)n1cc(C(=O)O)c(=O)cc1-2]

or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

Exemplary embodiments include compounds having the formula (XXI) or a pharmaceutically acceptable salt form thereof:

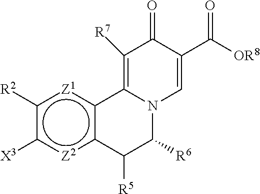

wherein non-limiting examples of X³. Z¹, Z², R², R⁵, R⁶, R⁷, and R⁸ are defined herein below in Table 1: Exemplary compounds of the formula ((XXI)

| Entry | X³ | Z¹ | Z² | R² | R⁵ | R⁶ | R⁷ | R⁸ |
|---|---|---|---|---|---|---|---|---|
| 1 | 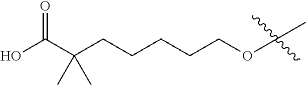 [SMILES: *OCCCCCC(C)(C)C(=O)O] | CH | CH | OMe | H | i-propyl | H | H |
| 2 | 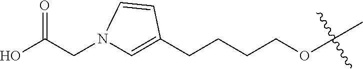 [SMILES: *OCCCCc1ccn(CC(=O)O)c1] | CH | CH | OMe | H | i-propyl | H | H |
| 3 | 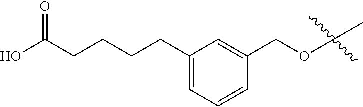 [SMILES: *OCc1cccc(CCCCC(=O)O)c1] | CH | CH | OMe | H | i-propyl | H | H |
| 4 | 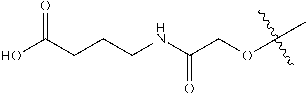 [SMILES: *OCC(=O)NCCCC(=O)O] | CH | CH | OMe | H | i-propyl | H | H |
| 5 | 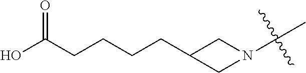 [SMILES: *N1CC(CCCCC(=O)O)C1] | CH | CH | OMe | H | i-propyl | H | H |
| 6 | 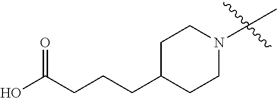 [SMILES: *N1CCC(CCCC(=O)O)CC1] | CH | CH | OMe | H | i-propyl | H | H |
| 7 | 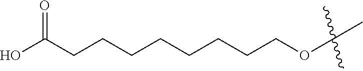 [SMILES: *OCCCCCCCCC(=O)O] | N | CH | OMe | H | i-propyl | H | H |
| 8 | 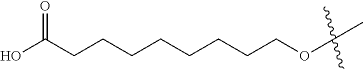 [SMILES: *OCCCCCCCCC(=O)O] | CH | N | OMe | H | i-propyl | H | H |
| 9 | 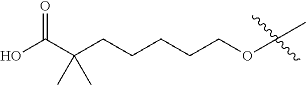 [SMILES: *OCCCCCC(C)(C)C(=O)O] | CH | CH | Cl | H | i-propyl | H | H |
| 10 | 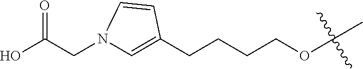 [SMILES: *OCCCCc1ccn(CC(=O)O)c1] | CH | CH | Cl | H | i-propyl | H | H |
| 11 | 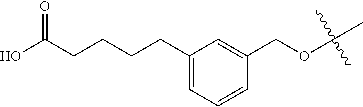 [SMILES: *OCc1cccc(CCCCC(=O)O)c1] | CH | CH | Cl | H | i-propyl | H | H |
| 12 | 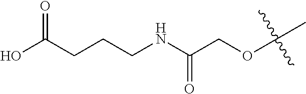 [SMILES: *OCC(=O)NCCCC(=O)O] | CH | CH | Cl | H | i-propyl | H | H |
| 13 | 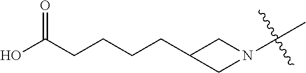 [SMILES: *N1CC(CCCCC(=O)O)C1] | CH | CH | Cl | H | i-propyl | H | H |
| 14 | 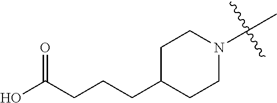 [SMILES: *N1CCC(CCCC(=O)O)CC1] | CH | CH | Cl | H | i-propyl | H | H |
| 15 | 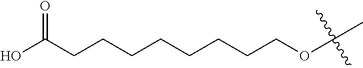 [SMILES: *OCCCCCCCCC(=O)O] | N | CH | Cl | H | i-propyl | H | H |
| 16 | 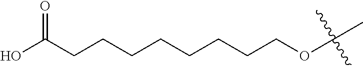 [SMILES: *OCCCCCCCCC(=O)O] | CH | N | Cl | H | i-propyl | H | H |
| 17 | 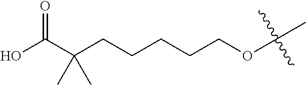 [SMILES: *OCCCCCC(C)(C)C(=O)O] | CH | CH | OMe | H | i-propyl | F | H |
| 18 | 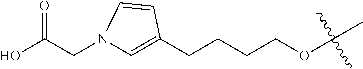 [SMILES: *OCCCCc1ccn(CC(=O)O)c1] | CH | CH | OMe | H | i-propyl | F | H |
| 19 | 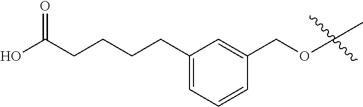 [SMILES: *OCc1cccc(CCCCC(=O)O)c1] | CH | CH | OMe | H | i-propyl | F | H |
| 20 | 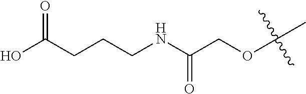 [SMILES: *OCC(=O)NCCCC(=O)O] | CH | CH | OMe | H | i-propyl | F | H |
| 21 | 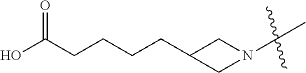 [SMILES: *N1CC(CCCCC(=O)O)C1] | CH | CH | OMe | H | i-propyl | F | H |
| 22 | 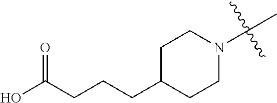 [SMILES: *N1CCC(CCCC(=O)O)CC1] | CH | CH | OMe | H | i-propyl | F | H |
| 23 | 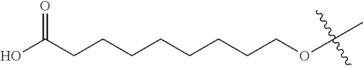 [SMILES: *OCCCCCCCCC(=O)O] | N | CH | OMe | H | i-propyl | F | H |
| 24 | 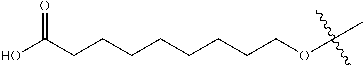 [SMILES: *OCCCCCCCCC(=O)O] | CH | N | OMe | H | i-propyl | F | H |
| 25 | 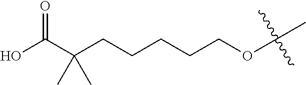 [SMILES: *OCCCCCC(C)(C)C(=O)O] | CH | CH | OMe | H | i-propyl | H | Et |
| 26 | 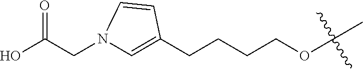 [SMILES: *OCCCCc1ccn(CC(=O)O)c1] | CH | CH | OMe | H | i-propyl | H | Et |
| 27 | 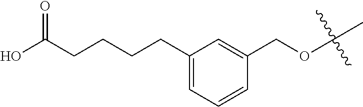 [SMILES: *OCc1cccc(CCCCC(=O)O)c1] | CH | CH | OMe | H | i-propyl | H | Et |
| 28 | 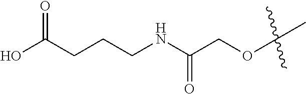 [SMILES: *OCC(=O)NCCCC(=O)O] | CH | CH | OMe | H | i-propyl | H | Et |
| 29 | 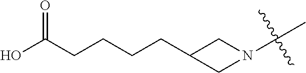 [SMILES: *N1CC(CCCCC(=O)O)C1] | CH | CH | OMe | H | i-propyl | H | Et |
| 30 | 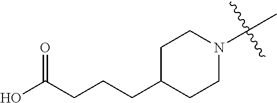 [SMILES: *N1CCC(CCCC(=O)O)CC1] | CH | CH | OMe | H | i-propyl | H | Et |
| 31 | 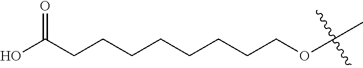 [SMILES: *OCCCCCCCCC(=O)O] | N | CH | OMe | H | i-propyl | H | Et |
| 32 | 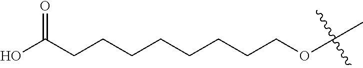 [SMILES: *OCCCCCCCCC(=O)O] | CH | N | OMe | H | i-propyl | H | Et |

Exemplary embodiments include compounds having the formula (XXII) or a pharmaceutically acceptable salt form thereof:

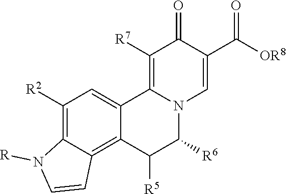

wherein non-limiting examples of R, R², R⁵, R⁶, R⁷, and R⁸ are defined herein below in Table 2.

TABLE 2
Exemplary compound of the formula (XXII)

| Entry | R | R² | R⁵ | R⁶ | R⁷ | R⁸ |
|---|---|---|---|---|---|---|
| 1 | 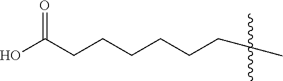 [SMILES: *CCCCCCC(=O)O] | OMe | H | i-propyl | H | H |
| 2 | 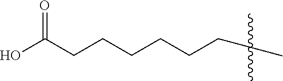 [SMILES: *CCCCCCC(=O)O] | OMe | H | i-propyl | H | H |
| 3 | 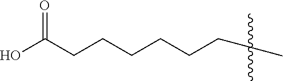 [SMILES: *CCCCCCC(=O)O] | Cl | H | i-propyl | H | H |
| 4 | 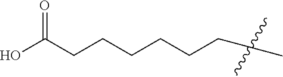 [SMILES: *CCCCCCC(=O)O] | OMe | H | i-propyl | H | Et |

Exemplary embodiments include compounds having the formula (XXIII) or a pharmaceutically acceptable salt form thereof:

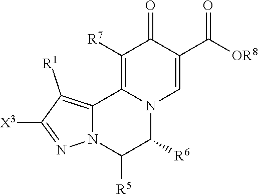

wherein non-limiting examples of X³, R¹, R⁵, R⁶, R⁷, and R⁸ are defined herein below in Table 3.

TABLE 3
Exemplary compound of the formula (XXIII)

| Entry | X³ | R¹ | R⁵ | R⁶ | R⁷ | R⁸ |
|---|---|---|---|---|---|---|
| 1 | 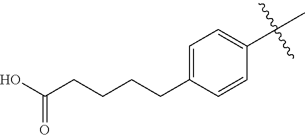 [SMILES: *c1ccc(CCCCC(=O)O)cc1] | H | H | t-butyl | F | H |
| 2 | 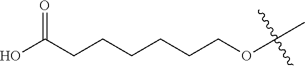 [SMILES: *OCCCCCCC(=O)O] | H | H | t-butyl | F | H |
| 3 | 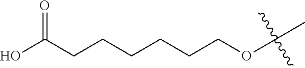 [SMILES: *OCCCCCCC(=O)O] | H | H | t-butyl | F | H |
| 4 | 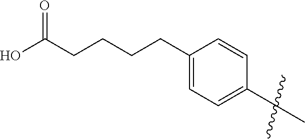 [SMILES: *c1ccc(CCCCC(=O)O)cc1] | H | H | t-butyl | H | Et |
| 5 | 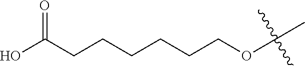 [SMILES: *OCCCCCCC(=O)O] | H | H | t-butyl | H | Et |
| 6 | 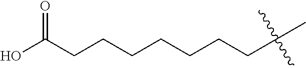 [SMILES: *CCCCCCCC(=O)O] | H | H | t-butyl | H | Et |
| 7 | 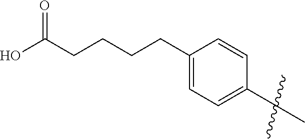 [SMILES: *c1ccc(CCCCC(=O)O)cc1] | H | H | t-butyl | H | H |
| 8 | 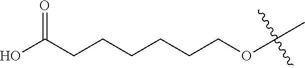 [SMILES: *OCCCCCCC(=O)O] | H | H | t-butyl | H | H |
| 9 | 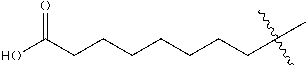 [SMILES: *CCCCCCCC(=O)O] | H | H | t-butyl | H | H |
| 10 | 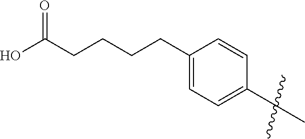 [SMILES: *c1ccc(CCCCC(=O)O)cc1] | F | H | t-butyl | H | H |
| 11 | 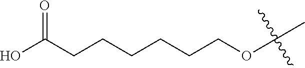 [SMILES: *OCCCCCCC(=O)O] | F | H | t-butyl | H | H |
| 12 | 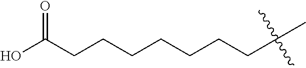 [SMILES: *CCCCCCCC(=O)O] | F | H | t-butyl | H | H |

Exemplary embodiments include compounds having the formula (XXIV) or a pharmaceutically acceptable salt form thereof:

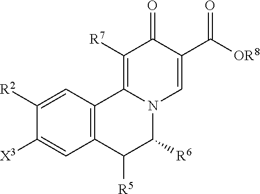

wherein non-limiting examples of X³, R², R⁵, R⁶, R⁷, and R⁸ are defined herein below in Table 4.

TABLE 4
Exemplary compound of the formula (XXIV)

| Entry | X³ | R² | R⁵ | R⁶ | R⁷ | R⁸ |
|---|---|---|---|---|---|---|
| 1 | —OCH₂CO₂H | OMe | H | i-propyl | H | H |
| 2 | —O(CH₂)₃CO₂H | OMe | H | i-propyl | H | H |
| 3 | —O(CH₂)₄CO₂H | OMe | H | i-propyl | H | H |
| 4 | —O(CH₂)₅CO₂H | OMe | H | i-propyl | H | H |
| 5 | —O(CH₂)₃OCH₂CO₂H | OMe | H | i-propyl | H | H |
| 6 | —O(CH₂)₆CO₂H | OMe | H | i-propyl | H | H |
| 7 | —O(CH₂)₇CO₂H | OMe | H | i-propyl | H | H |
| 8 | —O(CH₂)₈CO₂H | OMe | H | i-propyl | H | H |
| 9 | —OCH₂CO₂H | Cl | H | i-propyl | H | H |
| 10 | —O(CH₂)₃CO₂H | Cl | H | i-propyl | H | H |
| 11 | —O(CH₂)₄CO₂H | Cl | H | i-propyl | H | H |
| 12 | —O(CH₂)₅CO₂H | F | H | i-propyl | H | H |
| 13 | —O(CH₂)₃OCH₂CO₂H | F | H | i-propyl | H | H |
| 14 | —O(CH₂)₆CO₂H | F | H | i-propyl | H | H |
| 15 | —O(CH₂)₇CO₂H | F | H | i-propyl | H | H |
| 16 | —O(CH₂)₈CO₂H | F | H | i-propyl | H | H |
| 17 | —OCH₂CO₂H | OMe | H | i-propyl | Cl | H |
| 18 | —O(CH₂)₃CO₂H | OMe | H | i-propyl | Cl | H |
| 19 | —O(CH₂)₄CO₂H | OMe | H | i-propyl | Cl | H |
| 20 | —O(CH₂)₅CO₂H | OMe | H | i-propyl | Cl | H |
| 21 | —O(CH₂)₃OCH₂CO₂H | OMe | H | i-propyl | Cl | H |
| 22 | —O(CH₂)₆CO₂H | OMe | H | i-propyl | F | H |
| 23 | —O(CH₂)₇CO₂H | OMe | H | i-propyl | F | H |
| 24 | —O(CH₂)₈CO₂H | OMe | H | i-propyl | F | H |
| 25 | —OCH₂CO₂H | OMe | H | i-propyl | H | Et |
| 26 | —O(CH₂)₃CO₂H | OMe | H | i-propyl | H | Et |
| 27 | —O(CH₂)₄CO₂H | OMe | H | i-propyl | H | Et |
| 28 | —O(CH₂)₅CO₂H | OMe | H | i-propyl | H | Et |
| 29 | —O(CH₂)₃OCH₂CO₂H | OMe | H | i-propyl | H | Et |
| 30 | —O(CH₂)₆CO₂H | OMe | H | i-propyl | H | Et |
| 31 | —O(CH₂)₇CO₂H | OMe | H | i-propyl | H | Et |
| 32 | —O(CH₂)₈CO₂H | OMe | H | i-propyl | H | Et |

Exemplary embodiments include compounds having the formula (XXVI) or a pharmaceutically acceptable salt form thereof:

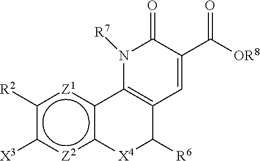

wherein non-limiting examples of X³, X⁴, Z¹, Z², R², R⁶, R⁷, and R⁸ are defined herein below in Table 5.

TABLE 5
Exemplary compound of the formula (XXVII)

| Entry | X³ | X⁴ | Z¹ | Z² | R² | R⁶ | R⁷ | R⁸ |
|---|---|---|---|---|---|---|---|---|
| 1 | —O(CH₂)₆CO₂H | CH₂ | CH | CH | Cl | i-propyl | H | H |
| 2 | —O(CH₂)₆CO₂H | SO₂ | CH | CH | Cl | i-propyl | Cyc- | H |
| | | | | | | | propyl | |
| 3 | —O(CH₂)₈CO₂H | O | CH | CH | Cl | i-propyl | Me | H |
| 4 | —O(CH₂)₆CO₂H | CH₂ | CH | CH | OMe | i-propyl | H | H |
| 5 | —O(CH₂)₆CO₂H | SO₂ | CH | CH | OMe | i-propyl | Cyc- | H |
| | | | | | | | propyl | |
| 6 | —O(CH₂)₈CO₂H | O | CH | CH | OMe | i-propyl | Me | H |
| 7 | —O(CH₂)₆CO₂H | CH₂ | CH | CH | OMe | i-propyl | H | H |
| 8 | —O(CH₂)₆CO₂H | SO₂ | CH | CH | OMe | i-propyl | Cyc- | H |
| | | | | | | | propyl | |
| 9 | —O(CH₂)₆CO₂H | CH₂ | CH | CH | OMe | i-propyl | H | Et |
| 10 | —O(CH₂)₆CO₂H | SO₂ | CH | CH | OMe | i-propyl | Cyc- | Et |
| | | | | | | | propyl | |
| 11 | —O(CH₂)₈CO₂H | O | CH | CH | OMe | i-propyl | Me | Et |

### Process

The present invention further relates to a process for preparing the compounds of the disclosure.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

### General Synthetic Schemes for Preparation of Compounds

The reagents used in the preparation of the compounds of this invention can be either commercially obtained or can be prepared by standard procedures described in the literature. In accordance with this invention, compounds in the genus may be produced by one of the following reaction schemes.

The first aspect of the process of the present invention relates to a process for preparing novel compounds of the disclosure having the formula (I). Compounds of formula (I) may be prepared according to the process outlined in Schemes 1-41.

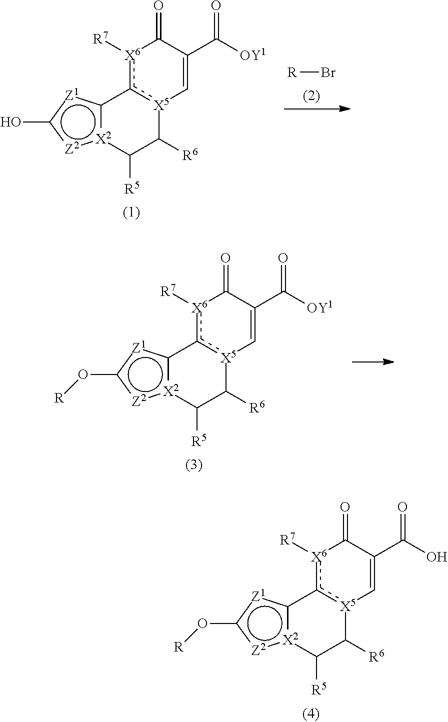

A compound of the formula (1), a known compound or a compound prepared by known methods in which Y¹ is a C₁₋₆alkyl, is reacted with a compound of the formula (2), a known compound or compound prepared by known methods, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (3). A compound of the formula (3) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (4).

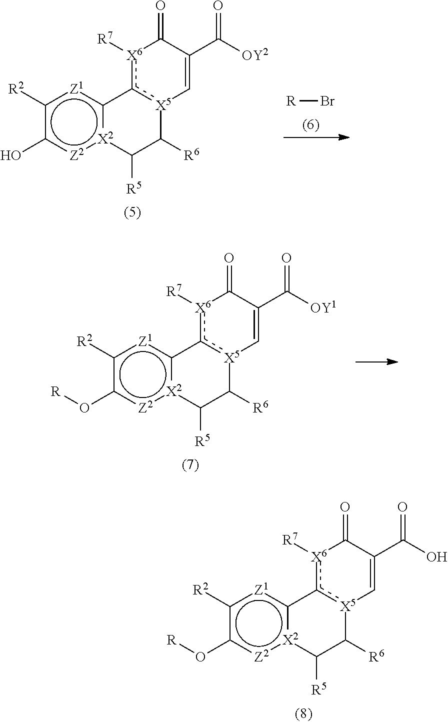

A compound of the formula (5), a known compound or a compound prepared by known methods in which Y² is a C₁₋₆alkyl, is reacted with a compound of the formula (6), a known compound or compound prepared by known methods, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (7). A compound of the formula (7) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (8).

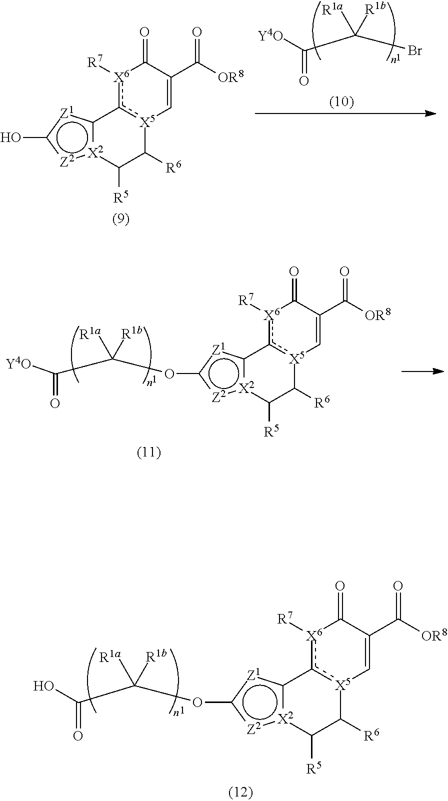

A compound of the formula (9), a known compound or a compound prepared by known methods, is reacted with a compound of the formula (10), a known compound or compound prepared by known methods in which Y⁴ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (11). A compound of the formula (11) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (12). Alternatively, a compound of the formula (11) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (12).

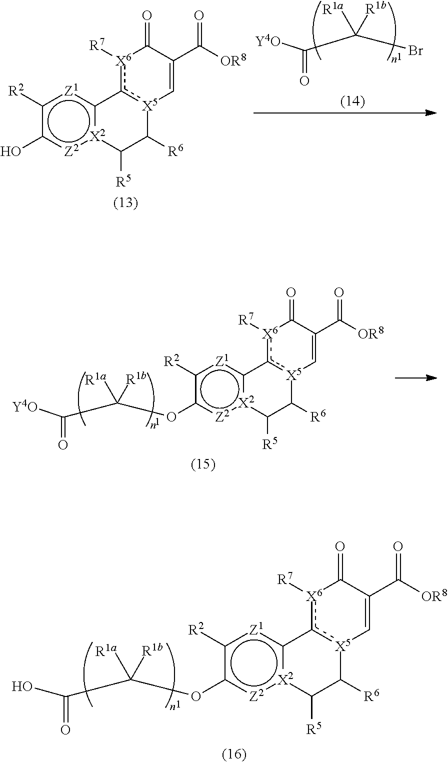

A compound of the formula (13), a known compound or a compound prepare by known methods, is reacted with a compound of the formula (14), a known compound or compound prepared by known methods in which Y⁴ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (15). A compound of the formula (15) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (16). Alternatively, a compound of the formula (15) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (16).

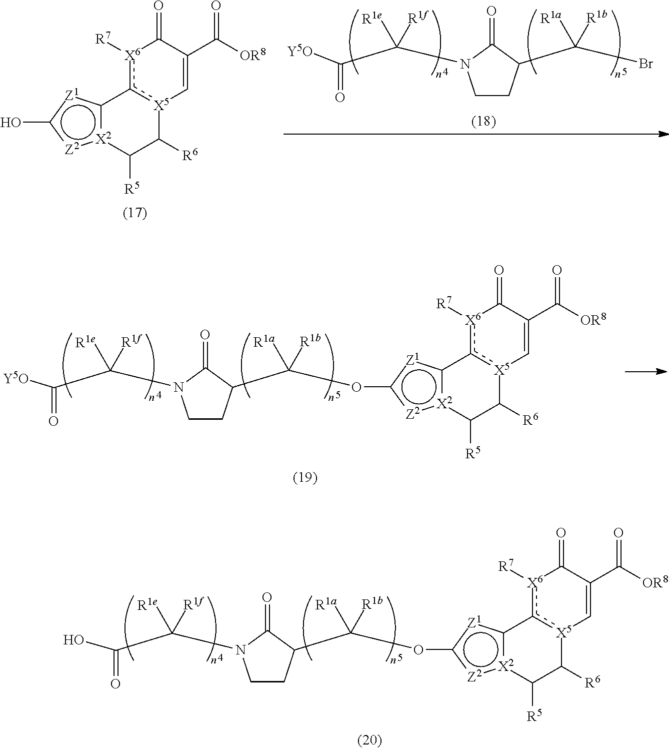

A compound of the formula (17), a known compound or a compound prepared by known methods, is reacted with a compound of the formula (18), a known compound or compound prepared by known methods in which Y⁵ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (19). A compound of the formula (19) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (20). Alternatively, a compound of the formula (19) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (20).

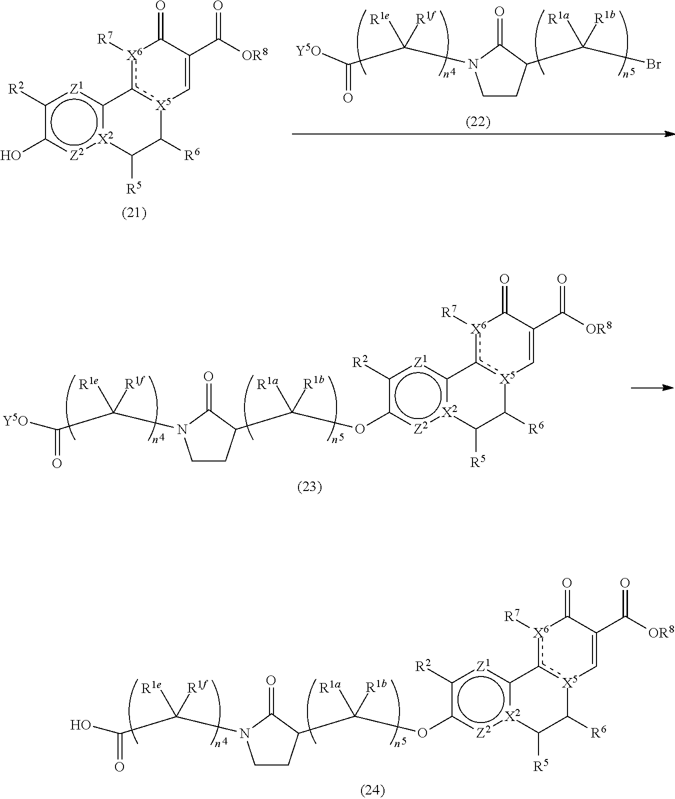

A compound of the formula (21), a known compound or a compound prepared by known methods, is reacted with a compound of the formula (22), a known compound or compound prepared by known methods in which Y⁵ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (23). A compound of the formula (23) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (24). Alternatively, a compound of the formula (23) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (24).

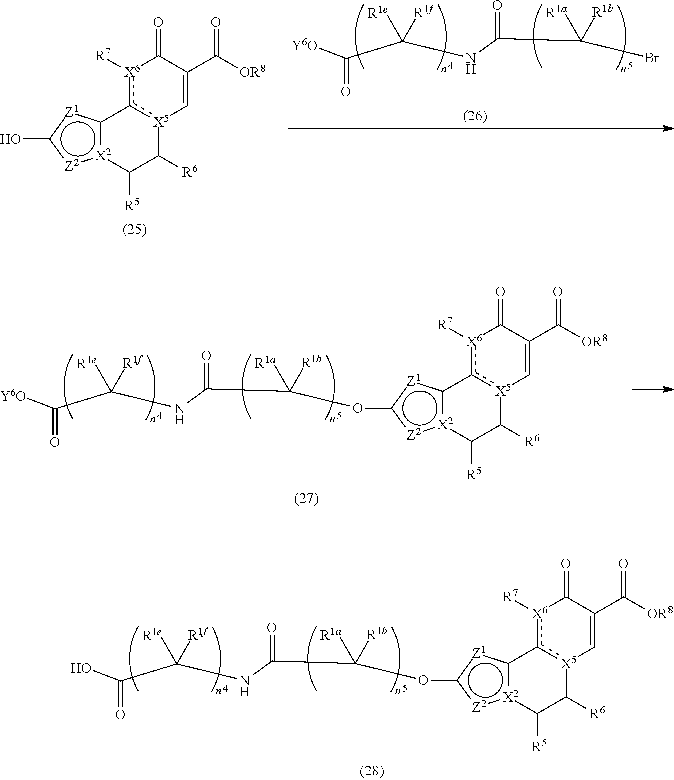

A compound of the formula (25), a known compound or a compound prepared by known methods, is reacted with a compound of the formula (26), a known compound or compound prepared by known methods in which Y⁶ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (27). A compound of the formula (27) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (28). Alternatively, a compound of the formula (27) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (28).

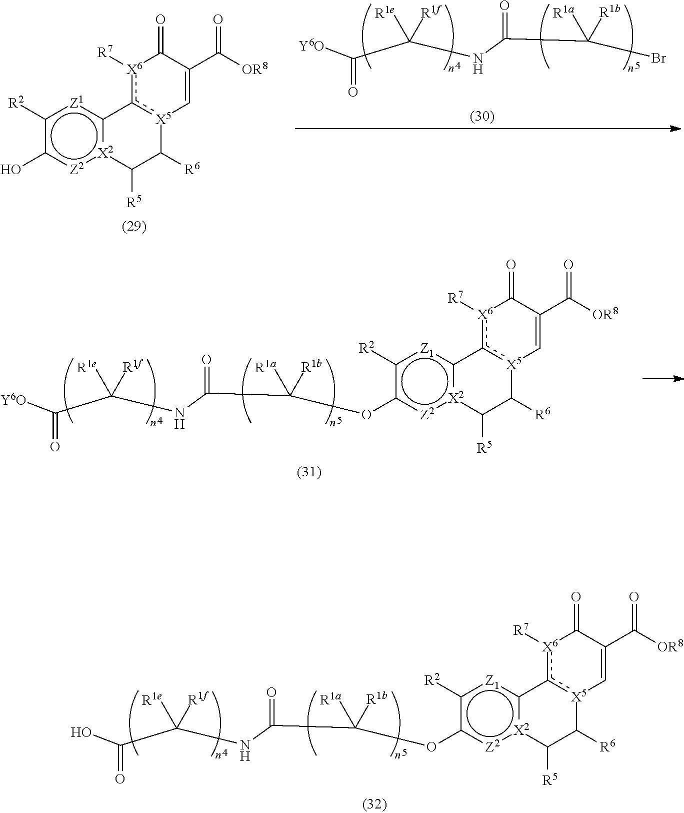

A compound of the formula (29), a known compound or a compound prepared by known methods, is reacted with a compound of the formula (30), a known compound or compound prepared by known methods in which Y⁶ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (31). A compound of the formula (31) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (32). Alternatively, a compound of the formula (31) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (32).

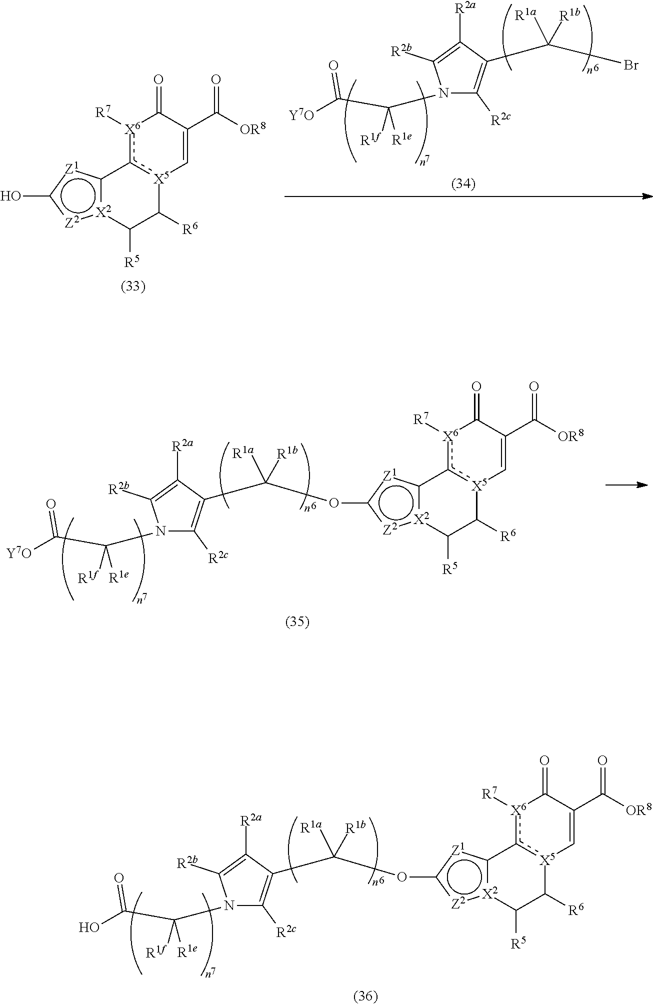

A compound of the formula (33), a known compound or a compound prepared by known methods, is reacted with a compound of the formula (34), a known compound or compound prepared by known methods in which Y⁷ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (35). A compound of the formula (35) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (36). Alternatively, a compound of the formula (35) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (36).

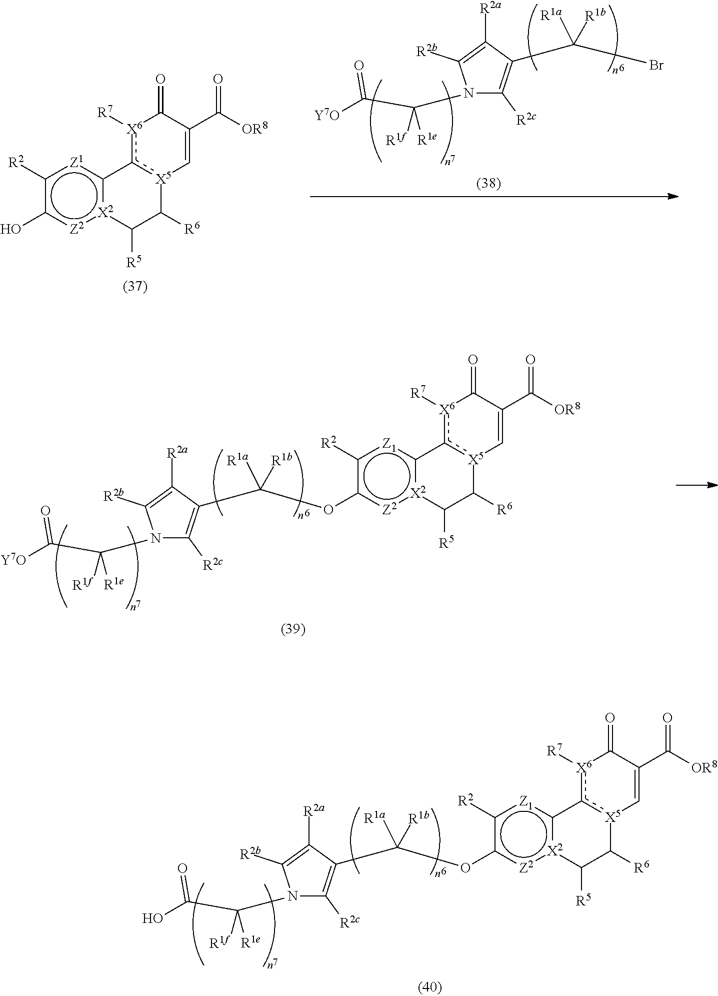

A compound of the formula (37), a known compound or a compound prepared by known methods, is reacted with a compound of the formula (38), a known compound or compound prepared by known methods in which Y⁷ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (39). A compound of the formula (39) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (40). Alternatively, a compound of the formula (39) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (40).

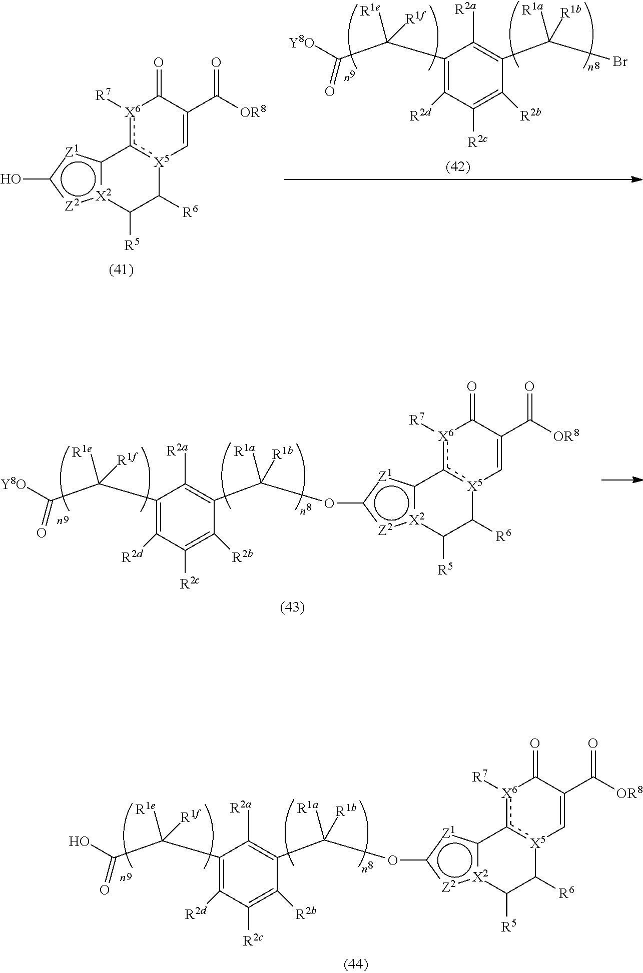

A compound of the formula (41), a known compound or a compound prepared by known methods, is reacted with a compound of the formula (42), a known compound or compound prepared by known methods in which Y⁸ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (43). A compound of the formula (43) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (44). Alternatively, a compound of the formula (43) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (44).

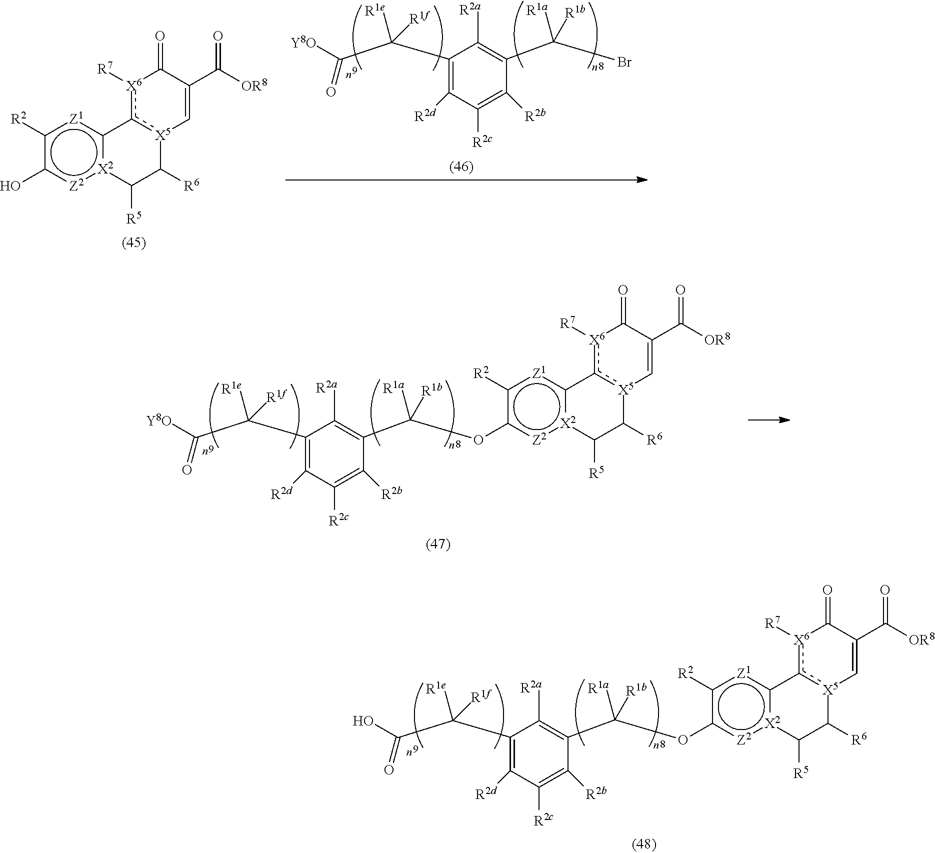

A compound of the formula (45), a known compound or a compound prepared by known methods, is reacted with a compound of the formula (46), a known compound or compound prepared by known methods in which Y⁸ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (47). A compound of the formula (47) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (48). Alternatively, a compound of the formula (47) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (48).

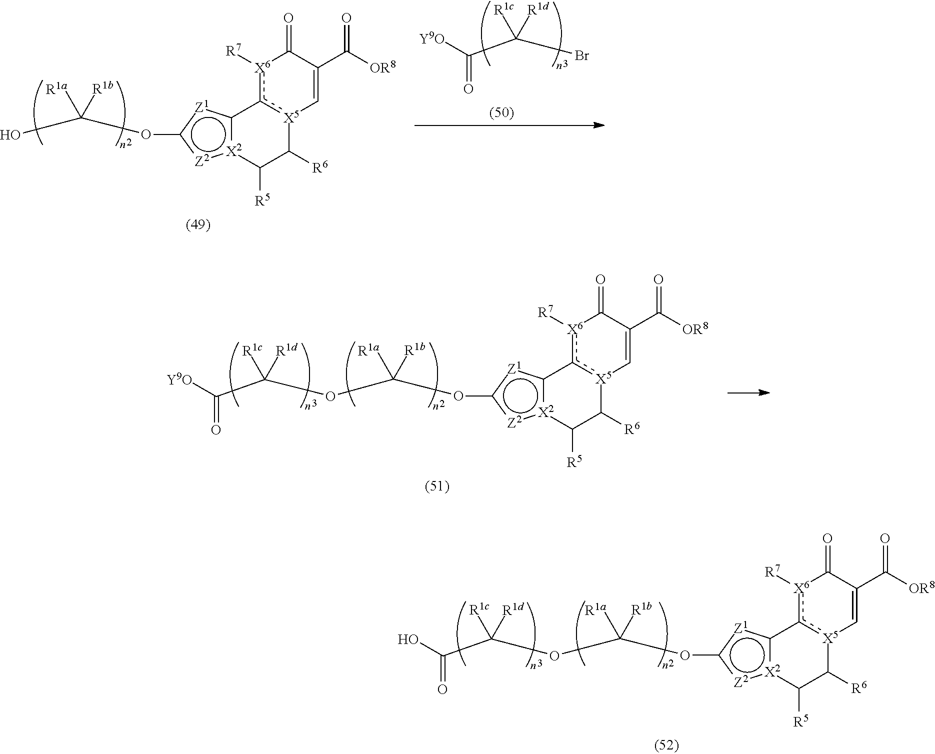

A compound of the formula (49), a known compound or a compound prepared by known methods, is reacted with a compound of the formula (50), a known compound or compound prepared by known methods in which Y⁹ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (51). A compound of the formula (51) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (52). Alternatively, a compound of the formula (51) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (52).

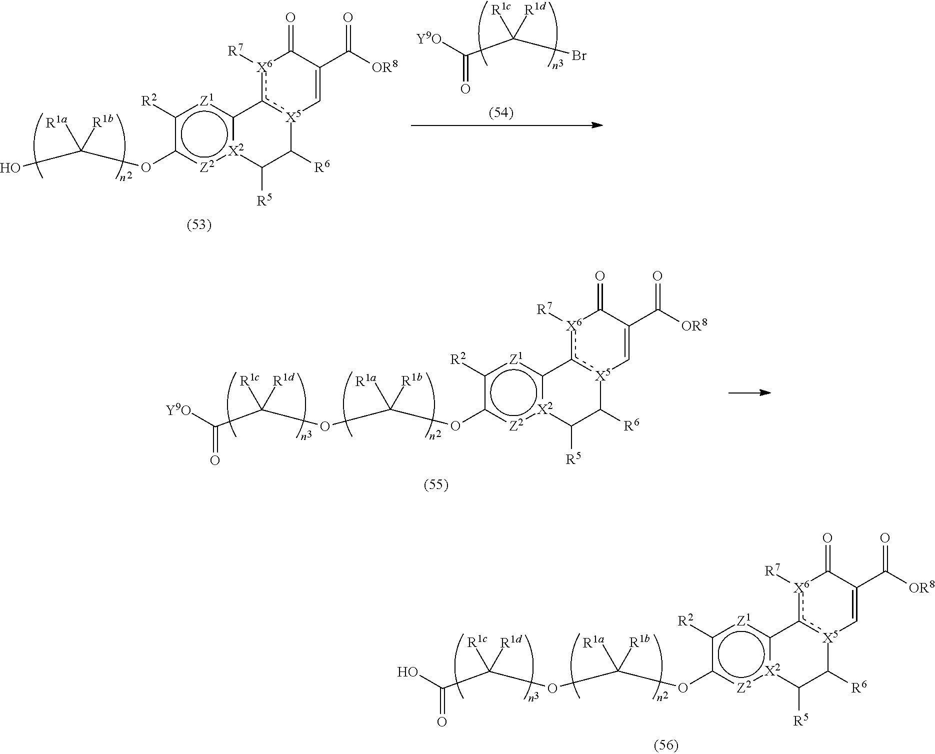

A compound of the formula (53), a known compound or a compound prepared by known methods, is reacted with a compound of the formula (54), a known compound or compound prepared by known methods in which Y⁹ is selected from the group consisting of C₁₋₆alkyl and tert-butyl, in the presence of a base such as sodium carbonate, potassium carbonate, lithium carbonate, triethylamine, diisopropylethylamine, pyridine, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (55). A compound of the formula (55) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (56). Alternatively, a compound of the formula (55) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (56).

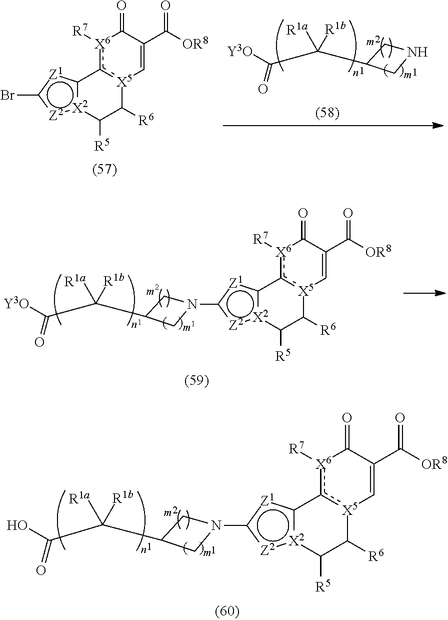

A compound of the formula (57), a known compound or a compound prepared by known methods, is reacted with a compound of the formula (58), a known compound or a compound prepared by known methods wherein Y³ is selected from the group consisting of C₁₋₆alkyl and tert-butyl, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′ triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (59). A compound of the formula (59) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (60). Alternatively, a compound of the formula (59) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (60).

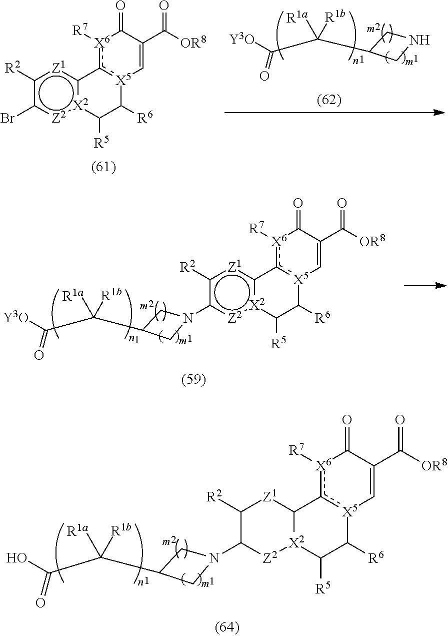

A compound of the formula (61), a known compound or a compound prepared by known methods, is reacted with a compound of the formula (62), a known compound or a compound prepared by known methods wherein Y³ is selected from the group consisting of C₁₋₆alkyl and tert-butyl, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (63). A compound of the formula (63) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (64). Alternatively, a compound of the formula (63) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (64).

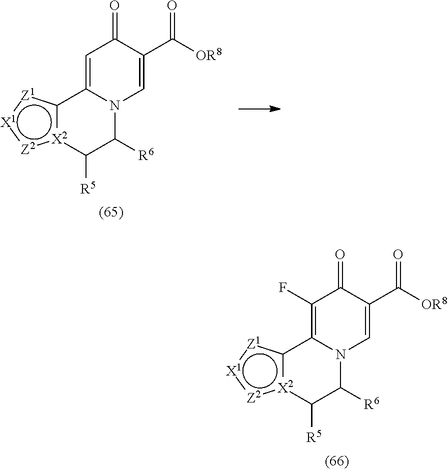

A compound of the formula (65), a known compound or a compound prepared by known methods, is reacted with 1-fluoro-4-methyl-1,4-diazoniabicyclo[2.2.2]octanebis(tetrafluoroborate) in the presence of a solvent such as ethanol, methanol, acetonitrile, dimethylformamide, dimethylacetamide, and the like, Optionally in the presence of an acid such as acetic acid, trifluoroacetic acid, hydrochloric acid, and the like, optionally in the presence of a base such as sodium carbonate, lithium carbonate, potassium carbonate, cesium carbonate, and the like. optionally with heating, optionally with microwave irradiation to provide a compound of the formula (66).

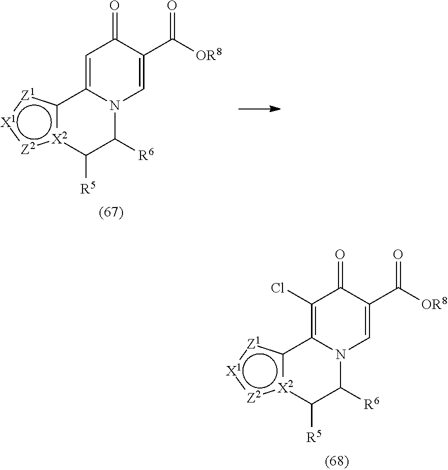

A compound of the formula (67), a known compound or a compound prepared by known methods, is reacted with trichloroisocyanuric acid in the presence of a solvent such as methanol, ethanol, methylene chloride, tetrahydrofuran, 1,4-dioxane, and the like, optionally with heating, optionally with microwave irradiation to provide a compound of the formula (68). Alternatively, a compound of the formula (67), a known compound or a compound prepared by known methods, is reacted with N-chlorosuccinimide in the presence of a solvent such as methanol, ethanol, methylene chloride, tetrahydrofuran, 1,4-dioxane, and the like, optionally with heating, optionally with microwave irradiation to provide a compound of the formula (68).

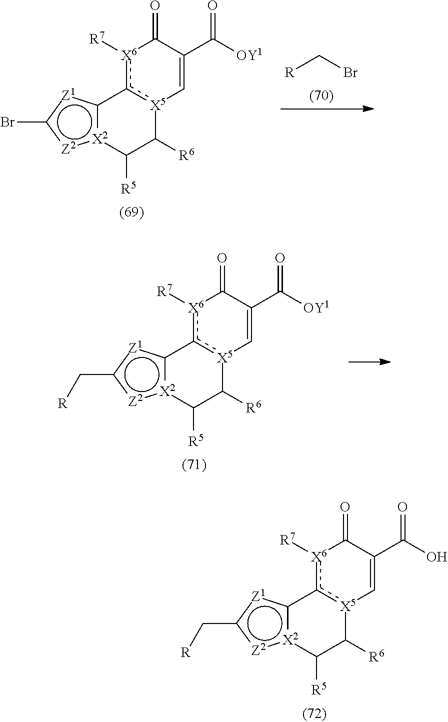

A compound of the formula (69), a known compound or a compound prepared by known methods where in Y¹ is a C₁₋₆alkyl, is reacted with a compound of the formula (70), in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (71). Alternatively, a compound of the formula (69), a known compound or a compound prepared by known methods where in Y¹ is a C₁₋₆alkyl, is reacted with a compound of the formula (70), in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (71). A compound of the formula (71) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (72).

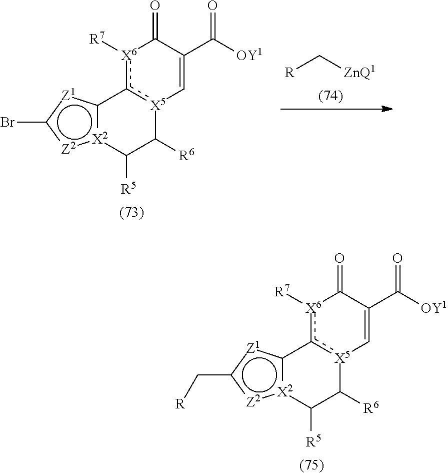

A compound of the formula (73), a known compound or a compound prepared by known methods where in Y¹ is a C₁₋₆alkyl and Q¹ is selected from the group consisting of halogen and (halogen)₂Li, is reacted with a compound of the formula (74), optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (75).

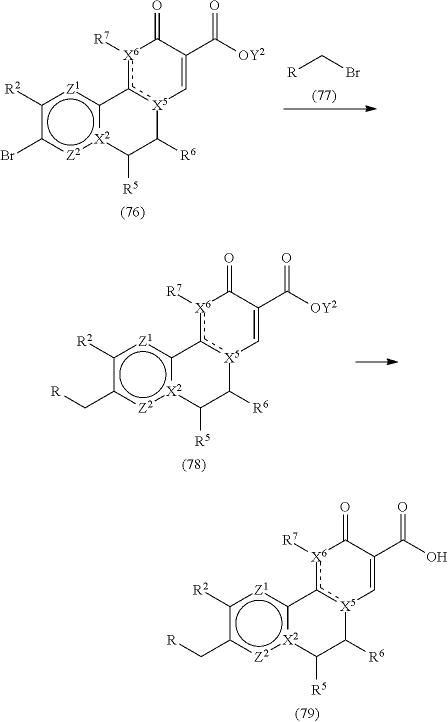

A compound of the formula (76), a known compound or a compound prepared by known methods in which Y² is a C₁₋₆alkyl, is reacted with a compound of the formula (77), a known compound or compound prepared by known methods, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (78). Alternatively, a compound of the formula (76), a known compound or a compound prepared by known methods where in Y² is a C₁₋₆alkyl, is reacted with a compound of the formula (77), in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (78). A compound of the formula (78) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (79).

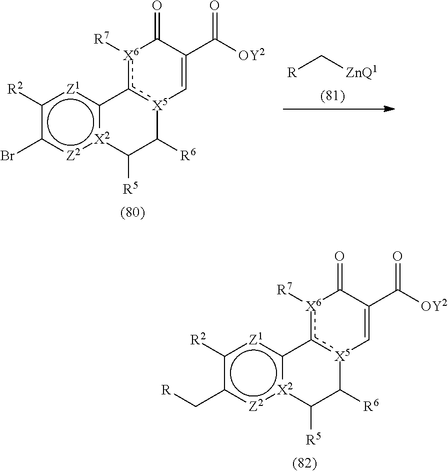

A compound of the formula (80), a known compound or a compound prepared by known methods where in Y² is a C₁₋₆alkyl and Q¹ is selected from the group consisting of halogen and (halogen)₂Li, is reacted with a compound of the formula (81), optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (82).

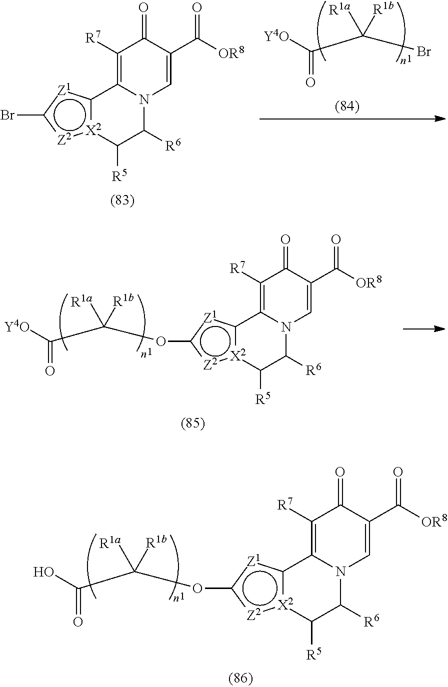

A compound of the formula (83), a known compound or a compound prepared by known methods, is reacted with a compound of the formula (84), a known compound or compound prepared by known methods in which Y⁴ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (85). Alternatively, a compound of the formula (83), a known compound or a compound prepared by known methods, is reacted with a compound of the formula (84), a known compound or compound prepared by known methods in which Y⁴ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (85). A compound of the formula (85) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (86). Alternatively, a compound of the formula (85) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (86).

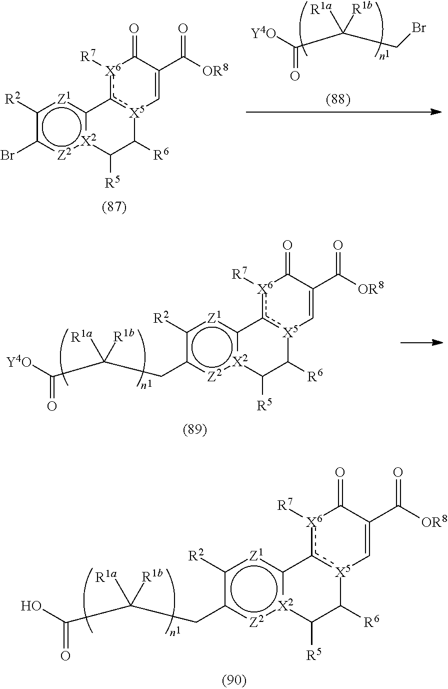

A compound of the formula (87), a known compound or a compound prepared by known methods, is reacted with a compound of the formula (88), a known compound or compound prepared by known methods in which Y⁴ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (89). Alternatively, compound of the formula (87), a known compound or a compound prepared by known methods, is reacted with a compound of the formula (88), a known compound or compound prepared by known methods in which Y⁴ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (89). A compound of the formula (89) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (90). Alternatively, a compound of the formula (89) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (90).

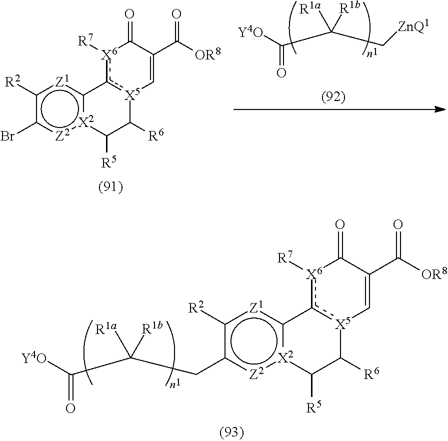

A compound of the formula (91), a known compound or a compound prepared by known methods, is reacted with a compound of the formula (92), a known compound or compound prepared by known methods in which Y⁴ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (93).

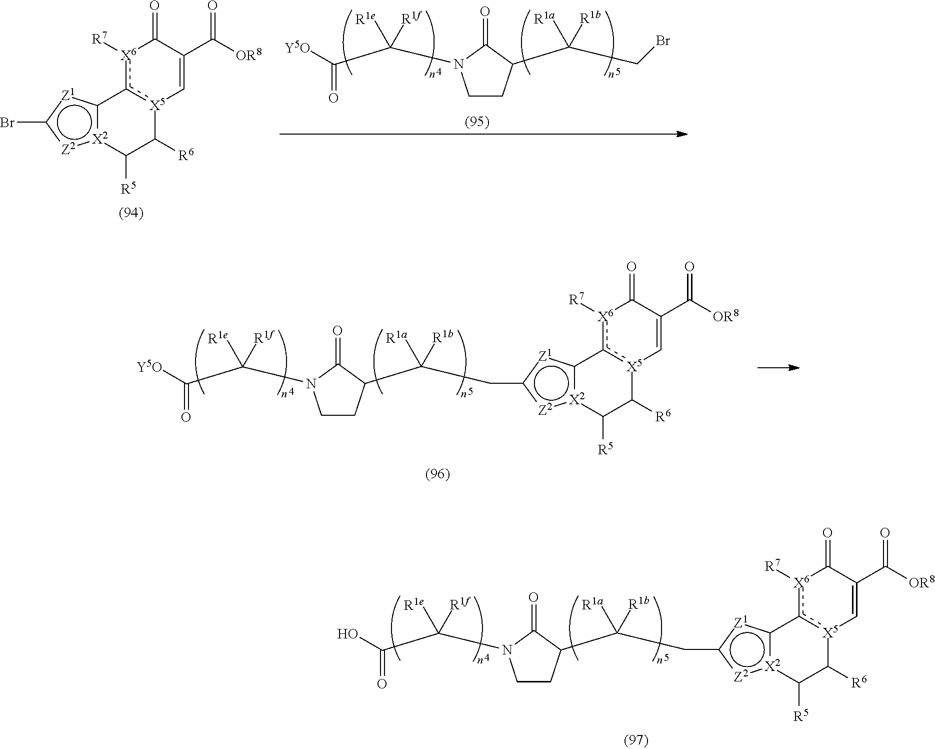

A compound of the formula (94), a known compound or a compound prepared by known methods, is reacted with a compound of the formula (95), a known compound or compound prepared by known methods in which Y⁵ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (96). Alternatively, a compound of the formula (94), a known compound or a compound prepared by known methods, is reacted with a compound of the formula (95), a known compound or compound prepared by known methods in which Y⁵ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (96). A compound of the formula (96) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (97). Alternatively, a compound of the formula (96) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (97).

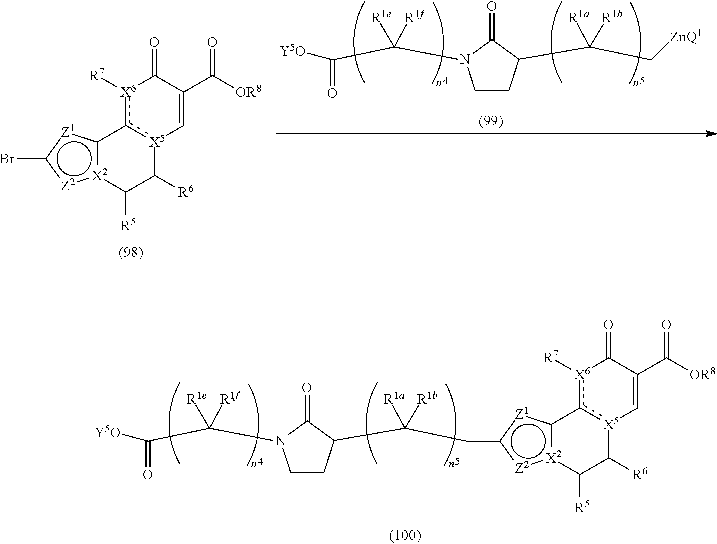

A compound of the formula (98), a known compound or a compound prepared by known methods, is reacted with a compound of the formula (99), a known compound or compound prepared by known methods in which Y⁵ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (100).

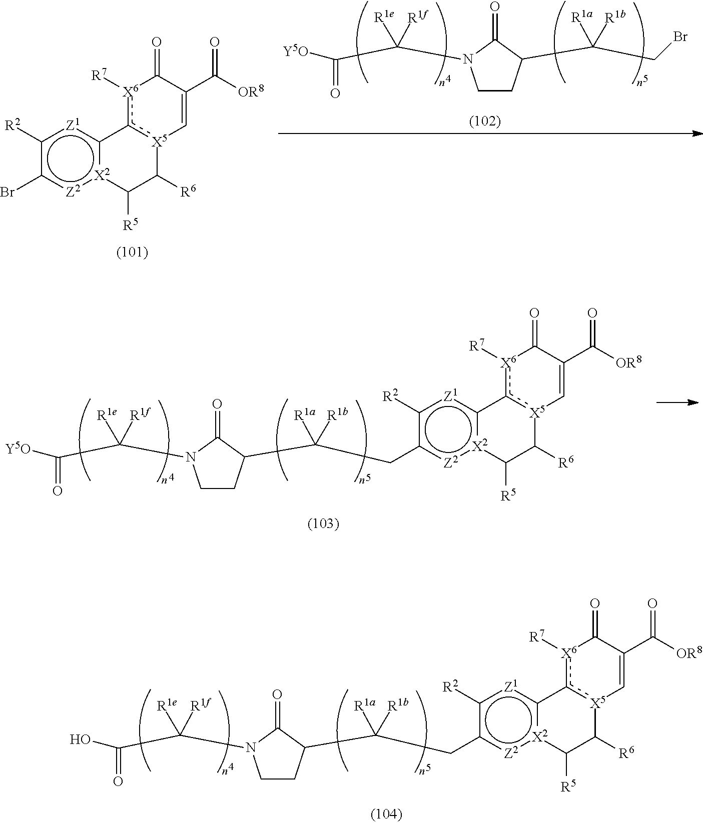

A compound of the formula (101), a known compound or a compound prepared by known methods, is reacted with a compound of the formula (102), a known compound or compound prepared by known methods in which Y⁵ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (103). Alternatively, a compound of the formula (101), is reacted with a compound of the formula (102), a known compound or compound prepared by known methods in which Y⁵ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (103). A compound of the formula (103) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (104). Alternatively, a compound of the formula (103) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (104).

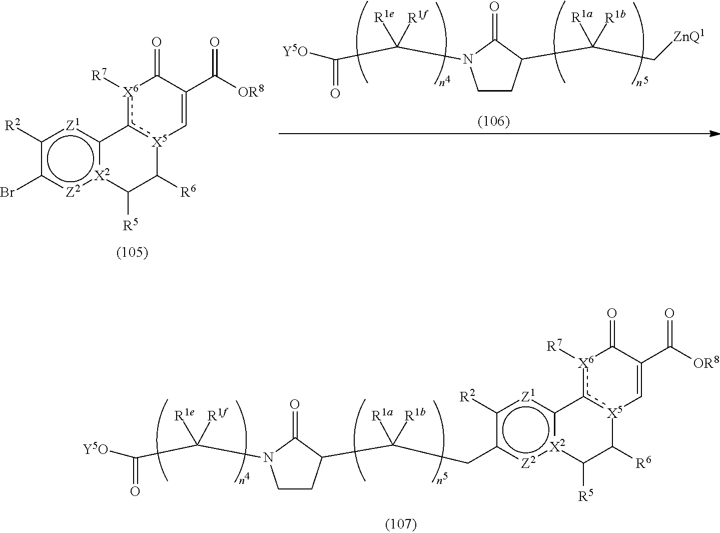

A compound of the formula (105), a known compound or a compound prepared by known methods, is reacted with a compound of the formula (106), a known compound or compound prepared by known methods in which Y⁵ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (107).

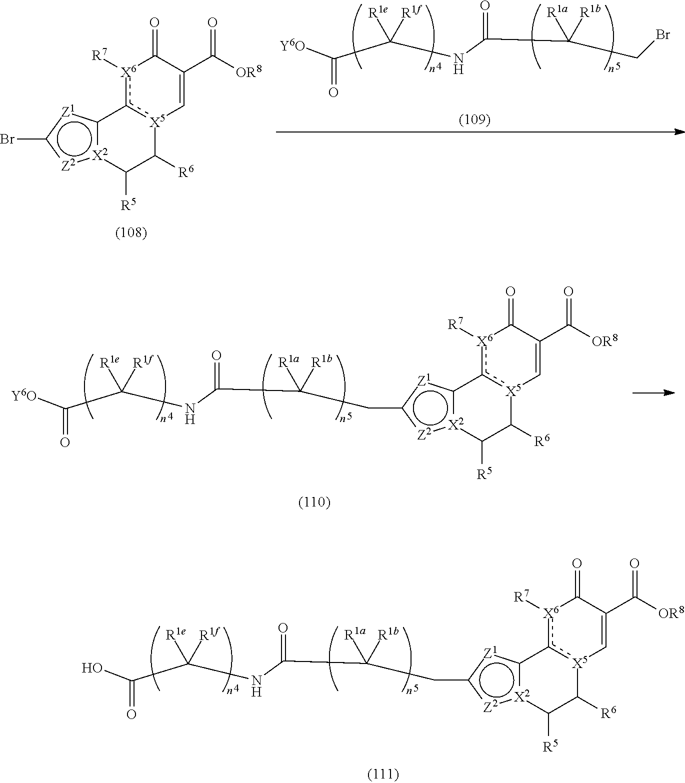

A compound of the formula (108), a known compound or a compound prepared by known methods, is reacted with a compound of the formula (109), a known compound or compound prepared by known methods in which Y⁶ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (110). Alternatively, a compound of the formula (108), a known compound or a compound prepared by known methods, is reacted with a compound of the formula (109), a known compound or compound prepared by known methods in which Y⁶ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (110). A compound of the formula (110) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (111). Alternatively, a compound of the formula (110) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (111).

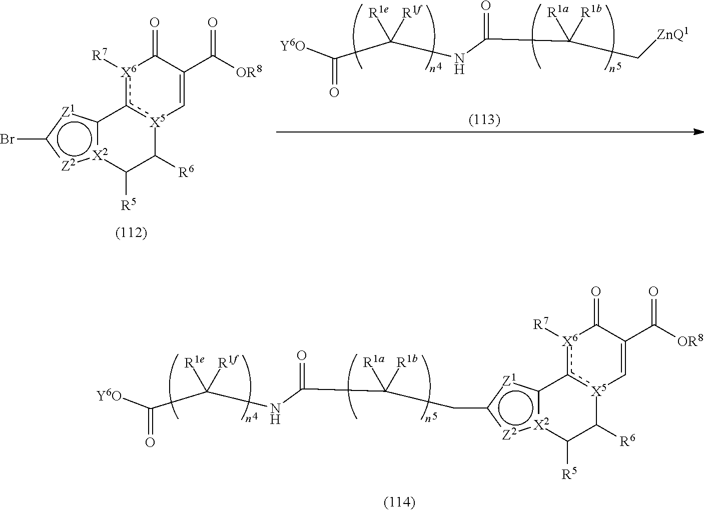

A compound of the formula (112), a known compound or a compound prepared by known methods, is reacted with a compound of the formula (113), a known compound or compound prepared by known methods in which Y⁶ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (114).

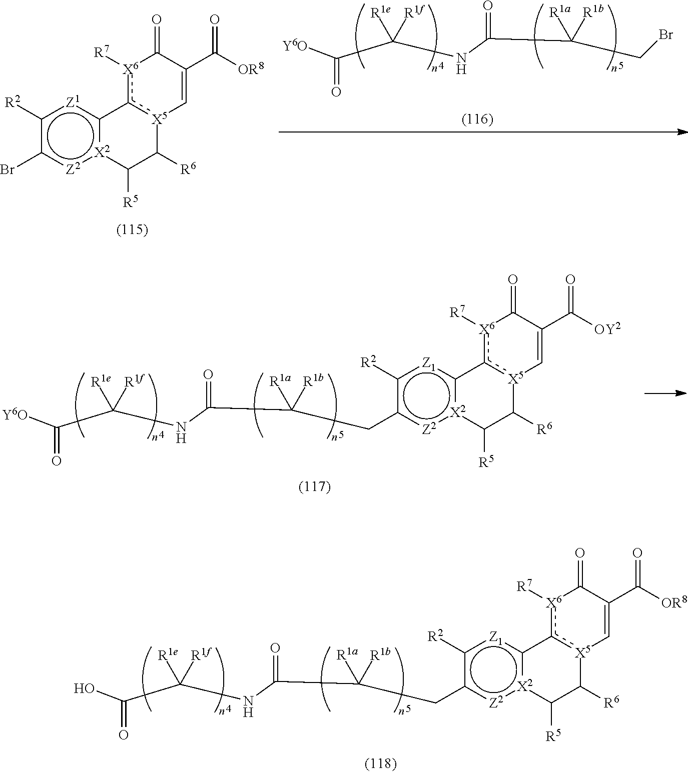

A compound of the formula (115), a known compound or a compound prepared by known methods, is reacted with a compound of the formula (116), a known compound or compound prepared by known methods in which Y⁶ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (117). Alternatively, a compound of the formula (115), a known compound or a compound prepared by known methods, is reacted with a compound of the formula (116), a known compound or compound prepared by known methods in which Y⁶ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (117). A compound of the formula (117) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (118). Alternatively, a compound of the formula (117) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (118).

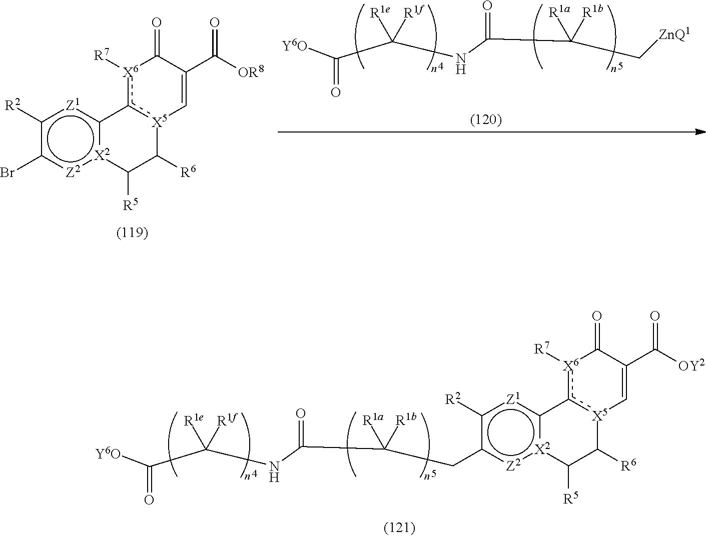

A compound of the formula (119), a known compound or a compound prepared by known methods, is reacted with a compound of the formula (120), a known compound or compound prepared by known methods in which Y⁶ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (121).

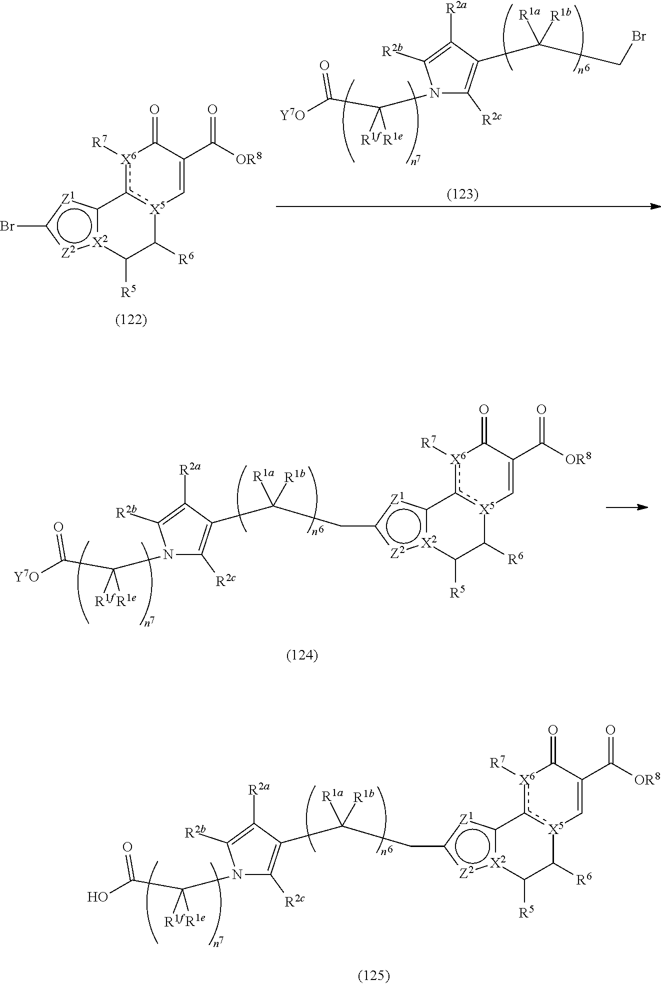

A compound of the formula (122), a known compound or a compound prepared by known methods, is reacted with a compound of the formula (123), a known compound or compound prepared by known methods in which Y⁷ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (124). Alternatively, a compound of the formula (122) is reacted with a compound of the formula (123), a known compound or compound prepared by known methods in which Y⁷ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (124). A compound of the formula (124) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (125). Alternatively, a compound of the formula (124) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (125).

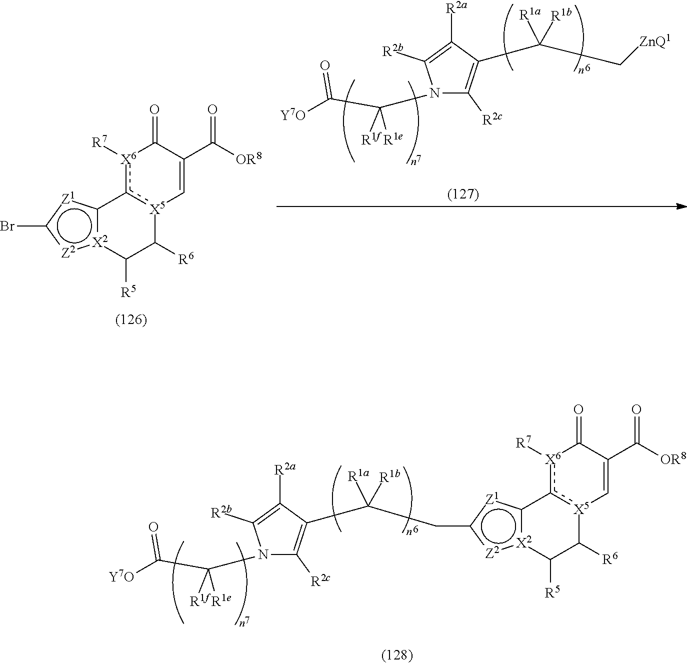

A compound of the formula (126), a known compound or a compound prepared by known methods, is reacted with a compound of the formula (127), a known compound or compound prepared by known methods in which Y⁷ is selected from the group consisting of C₁₋₆alkyl and tert-butyl and Q^{I} is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (128).

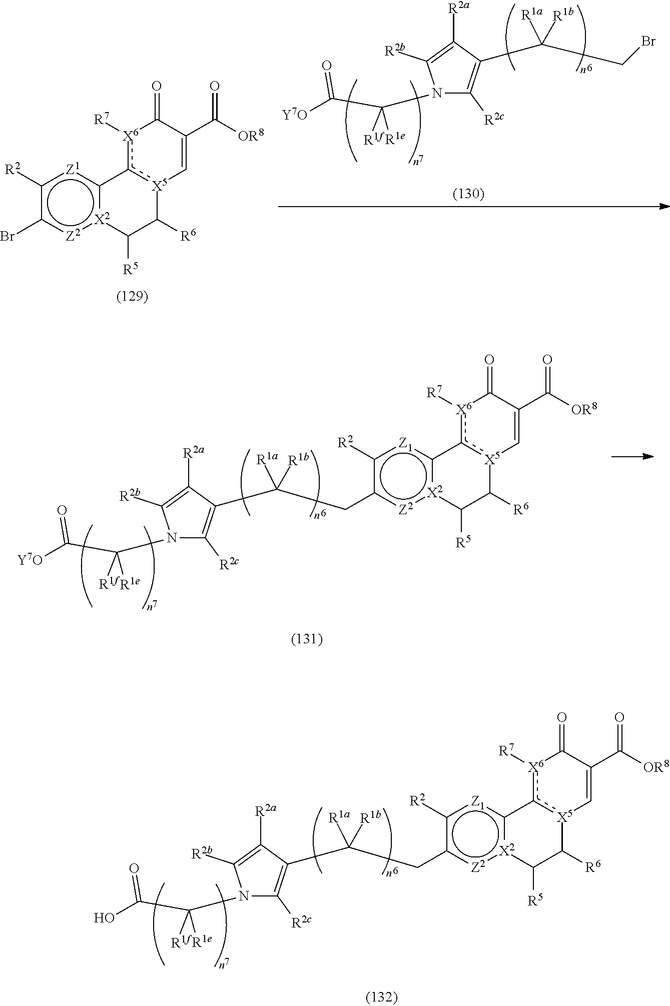

A compound of the formula (129), a known compound or a compound prepared by known methods, is reacted with a compound of the formula (130), a known compound or compound prepared by known methods in which Y⁷ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (131). Alternatively, a compound of the formula (129) is reacted with a compound of the formula (130), a known compound or compound prepared by known methods in which Y⁷ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′ triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (131). A compound of the formula (131) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (132). Alternatively, a compound of the formula (131) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (132).

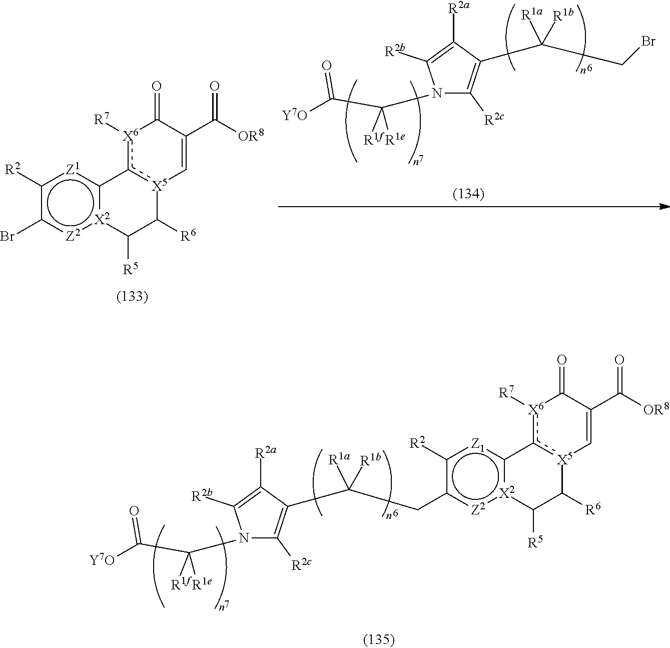

A compound of the formula (133) is reacted with a compound of the formula (134), a known compound or compound prepared by known methods in which Y⁷ is selected from the group consisting of C₁₋₆alkyl and tert-butyl, in the presence of zinc, in the presence of sodium iodide, in the presence of nickel(II) iodide, in the presence of 1,10-phenanthroline, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (135).

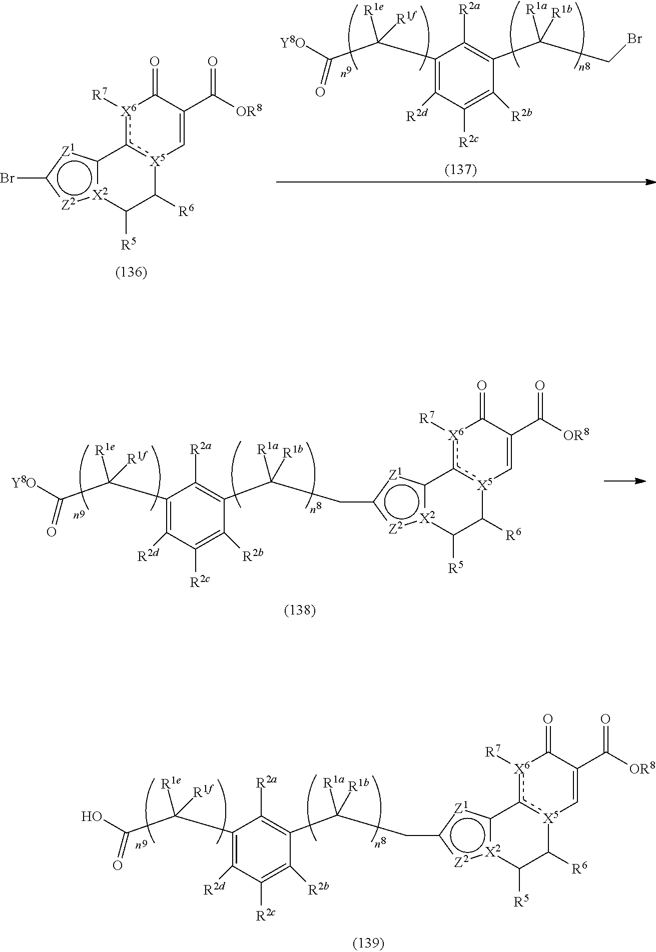

A compound of the formula (136), a known compound or a compound prepared by known methods, is reacted with a compound of the formula (137), a known compound or compound prepared by known methods in which Y⁸ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformamide, and the like, optionally with heating, optionally with microwave irradiation to provide a compound of the formula (138). Alternatively, a compound of the formula (136) is reacted with a compound of the formula (137), a known compound or compound prepared by known methods in which Y⁸ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (138). A compound of the formula (138) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (139). Alternatively, a compound of the formula (138) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (139).

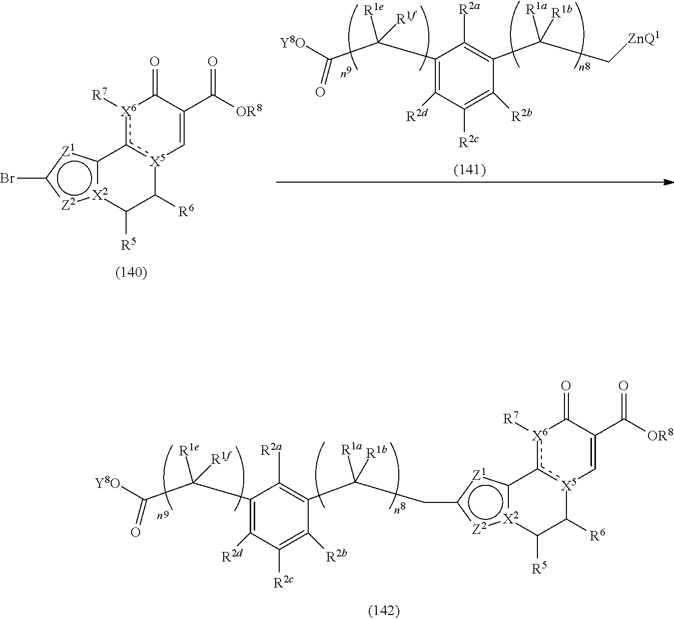

A compound of the formula (140) is reacted with a compound of the formula (141), a known compound or compound prepared by known methods in which Y⁸ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (142).

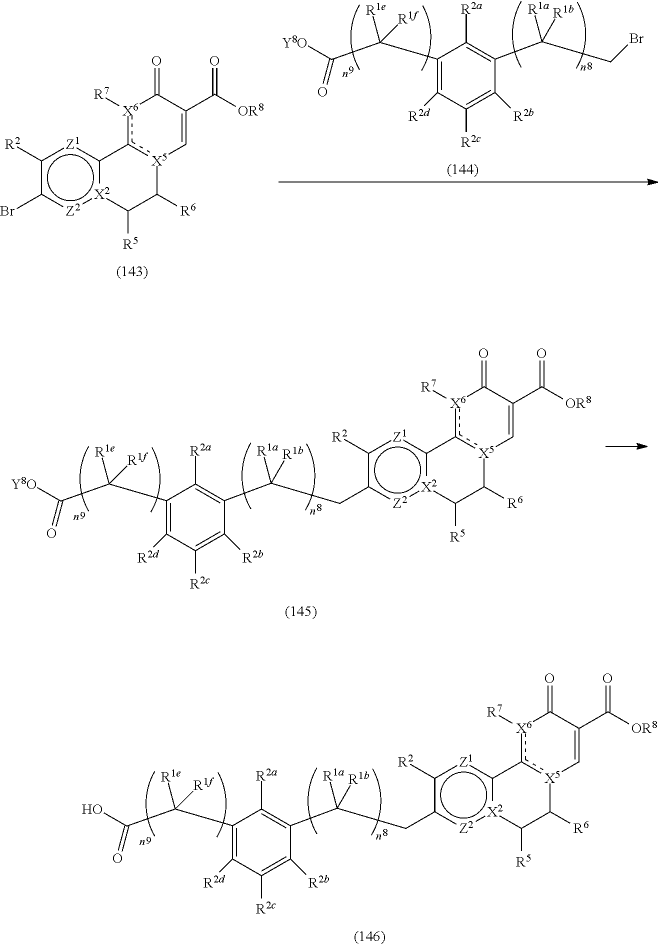

A compound of the formula (143) is reacted with a compound of the formula (144), a known compound or compound prepared by known methods in which Y⁸ is selected from the group consisting of C₁₋₆alkyl and tert-butyl, in the presence of zinc, in the presence of sodium iodide, in the presence of nickel(II) iodide, in the presence of 1,10-phenanthroline, in the presence of a solvent such as methylene chloride, tetrahydrofuran, 1,4-dioxane, dimethylformnamide, and the like, optionally with heating, optionally with microwave irradiation to provide a compound of the formula (145). Alternatively, a compound of the formula (143) is reacted with a compound of the formula (144), a known compound or compound prepared by known methods in which Y⁸ is selected from the group consisting of C₁₋₆alkyl and tert-butyl, in the presence of zinc, in the presence of a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′ (N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, in the presence of a base such as sodium carbonate, lithium carbonate, potassium carbonate, cesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (145). A compound of the formula (145) is reacted with a base such as sodium carbonate, potassium carbonate, lithium carbonate, lithium hydroxide, sodium hydroxide, potassium hydroxide, and the like in the presence of a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (146). Alternatively, a compound of the formula (145) is reacted with an acid such as trifluoroacetic acid, hydrochloric acid, sulfuric acid, and the like, in a solvent such as tetrahydrofuran, 1,4-dioxane, dimethylformamide, methylene chloride, dichloroethane, methanol, ethanol, and the like, optionally with heating, optionally with microwave irradiation to provide a compound of the formula (146).

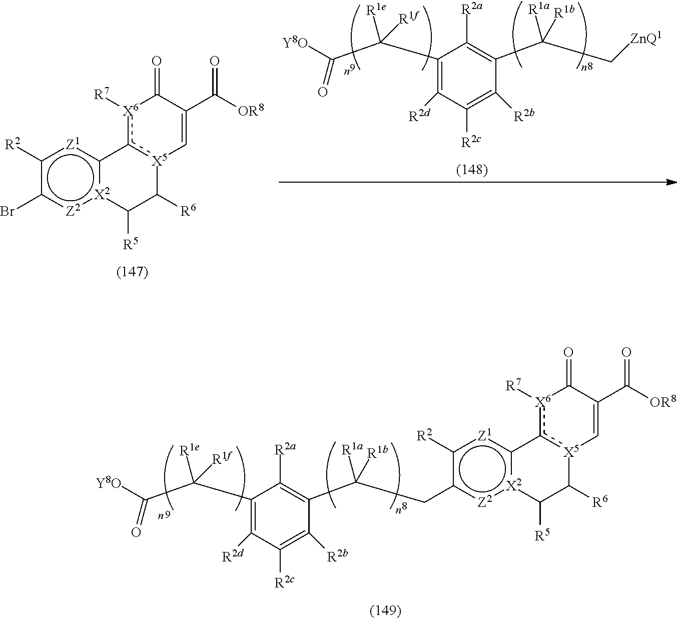

A compound of the formula (147) is reacted with a compound of the formula (148), a known compound or compound prepared by known methods in which Y⁸ is selected from the group consisting of C₁₋₆alkyl and tert-butyl and Q¹ is selected from the group consisting of halogen and (halogen)₂Li, optionally in the presence of sodium iodide, optionally in the presence of nickel(II) iodide, optionally in the presence of 1,10-phenanthroline, optionally in the presence of a palladium catalyst such as a palladium catalyst such as palladium (II) acetate, tetrakis(triphenylphosphine)palladium(0), dichlorobis (triphenylphosphine) palladium(II), palladium on carbon, bis(acetonitrile)dichloropalladium(II), and the like, optionally in the presence of a base such as pyridine, triethylamine, diisopropylethylamine, and the like, in the presence of a solvent such as methylene chloride, tetrahydrofuran, 1,4-dioxane, dimethylformamide, and the like, optionally with heating, optionally with microwave irradiation to provide a compound of the formula (149).

Additional methods necessary to prepare the compounds of the disclosure are described in WO2017016960, WO2019123285, WO2020106816A1, WO2018022282, WO2017216685, and WO2018130152 which are incorporated herein by reference.

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

¹H NMR spectra were recorded on a 300 MHz INOVA VARIAN spectrometer. Chemical shifts values are given in ppm and referred as the internal standard to TMS (tetramethylsilane). The peak patterns are indicated as follows: s, singlet; d, doublet; t, triplet; q, quadruplet; m, multiplet and dd, doublet of doublets. The coupling constants (J) are reported in Hertz (Hz). Mass Spectra were obtained on a Agilent 1200 LC-MS spectrometer (ES-API, Positive). Mobile phase flow was 1.0 mL/min with a 3.0 min gradient from 20% aqueous media (0.1% formic acid) to 95% CH₃CN (0.1% formic acid) and a 9.0 min total acquisition time. Silica gel column chromatography was performed using Teledyne ISCO silica gel columns (20-40 microns or 40-60 microns), and the eluent was a mixture of ethyl acetate and hexanes, or mixture of methanol and ethyl acetate. Preparative high-performance liquid chromatography (HPLC) was performed using a Gilson 331 and 332 pumps with a UV/VIS-155 detector and GX-271 liquid handler using a Phenomenex Luna LC Column (5 μm C18 100 Å, 150×21.2 mm). All the tested compounds possess a purity of at least 95%. Analytical HPLC was run on the Agilent 1100 HPLC instrument, equipped with Agilent, ZORBAX SB—C18 column and UV detection at 210 nm.

### EXAMPLES

Examples 1-8 provide methods for preparing representative compounds of formula (I). The skilled practitioner will know how to substitute the appropriate reagents, starting materials and purification methods known to those skilled in the art, in order to prepare additional compounds of the present invention.

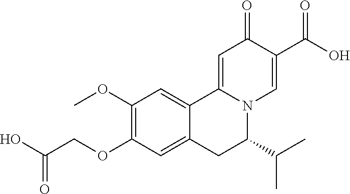 [SMILES: COc1cc2c(cc1OCC(=O)O)C[C@@H](C(C)C)n1cc(C(=O)O)c(=O)cc1-2]

### Example 1

Synthesis of (S)-9-(carboxymethoxy)-6-isopropyl-10-methoxy-2-oxo-6,7-dihydro-2H-pyrido [2,1-a]isoquinoline-3-carboxylic acid (65042-A-OH): (S)-ethyl 9-hydroxy-6-isopropyl-10-methoxy-2-oxo-6,7-dihydro-2H-pyrido[2,1-a]isoquinoline-3-carboxylate (15 mg, 0.0420 mmol) and potassium carbonate (17 mg, 0.126 mmol) was dissolved in 2 mL N,N-dimethylformamide. Methyl 2-bromoacetate (4 μL, 0.0462 mmol) was added, and the reaction was stirred for 18 hours. The reaction was partitioned between ethyl acetate and water, and then the aqueous phase was extracted with ethyl acetate thrice. The organic phases were combined, stripped of solvent, and the residue was purified by HPLC. The purified intermediate was dissolved in 2 mL tetrahydrofuran, and 3 eq. 1 M LiOH was added. After 2 hours, the reaction was concentrated down and acidified by 0.1% TFA in MeCN/H₂O, and then directly purified by HPLC to afford 65042-A-OH (12.6 mg, 77%). ¹H NMR (300 MHz, CDCl₃): δ (ppm) 8.44 (s, 1H), 7.21 (s, 1H), 7.06 (s, 1H), 6.67 (s, 1H), 4.71 (s, 2H), 3.93 (s, 3H), 3.91-3.68 (m, 1H), 3.31-3.23 (m, 1H), 3.06-2.99 (m, 1H), 1.80-1.70 (m, 1H), 0.89 (d, J=6.3 Hz, 3H), 0.78 (d, J=7.2 Hz, 3H); Calculated for C₂₀H₂₁NO₇, 387.13; observed (M+H)⁺ 388.8.

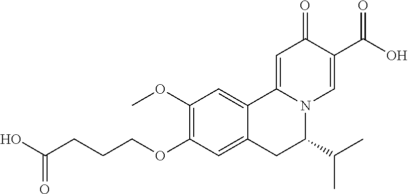 [SMILES: COc1cc2c(cc1OCCCC(=O)O)C[C@@H](C(C)C)n1cc(C(=O)O)c(=O)cc1-2]

### Example 2

Synthesis of (S)-9-(3-carboxypropoxy)-6-isopropyl-10-methoxy-2-oxo-6,7-dihydro-2H-pyrido [2,1-a]isoquinoline-3-carboxylic acid (65042-C—OH): (S)-9-(3-carboxypropoxy)-6-isopropyl-10-methoxy-2-oxo-6,7-dihydro-2H-pyrido [2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with methyl 4-bromobutanoate. ¹H NMR (300 MHz, CDCl₃): δ (ppm) 8.42 (s, 1H), 7.16 (s, 1H), 7.03 (s, 1H), 6.74 (s, 1H), 4.15-4.05 (m, 2H), 3.90-3.85 (m, 1H), 3.89 (s, 3H), 3.37-3.25 (m, 1H), 3.10-3.00 (m, 1H), 2.56-2.48 (m, 2H), 2.20-2.10 (m, 3H), 0.91 (d, J=6.6 Hz, 3H), 0.78 (d, J=6.3 Hz, 3H); Calculated for C₂₂H₂₅NO₇, 415.16; observed (M+H)⁺ 416.7.

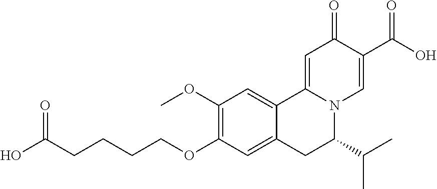 [SMILES: COc1cc2c(cc1OCCCCC(=O)O)C[C@@H](C(C)C)n1cc(C(=O)O)c(=O)cc1-2]

### Example 3

Synthesis of (S)-9-(4-carboxybutoxy)-6-isopropyl-10-methoxy-2-oxo-6,7-dihydro-2H-pyrido[2,1-a]isoquinoline-3-carboxylic acid (65042-D-OH): (S)-9-(4-carboxybutoxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with methyl 5-bromopentanoate. ¹H NMR (300 MHz, CDCl₃): δ (ppm) 8.44 (s, 1H), 7.16 (s, 1H), 7.04 (s, 1H), 6.71 (s, 1H), 4.10-4.03 (m, 2H), 3.90 (s, 3H), 3.88-3.82 (m, 1H), 3.35-3.26 (m, 1H), 3.07-3.01 (m, 1H), 2.42-2.36 (m, 2H), 1.95-1.74 (m, 5H), 0.92 (d, J=7.2 Hz, 3H), 0.79 (d, J=7.2 Hz, 3H); Calculated for C₂₃H₂₇NO₇, 429.18; observed (M+H)⁺ 430.7.

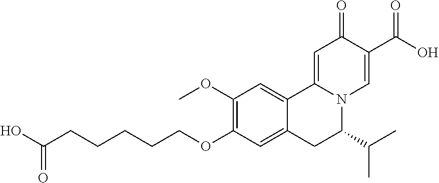 [SMILES: COc1cc2c(cc1OCCCCCC(=O)O)C[C@@H](C(C)C)n1cc(C(=O)O)c(=O)cc1-2]

### Example 4

Synthesis of (S)-9-((5-carboxypentyl)oxy)-6-isopropyl-10-methoxy-2-oxo-6,7-dihydro-2H-pyrido[2,1-a]isoquinoline-3-carboxylic acid (65025-B—OH): (S)-9-((5-carboxypentyl)oxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with ethyl 6-bromohexanoate. ¹H NMR (300 MHz, CDCl₃): δ (ppm) 8.43 (s, 1H), 7.13 (s, 1H), 7.02 (s, 1H), 6.69 (s, 1H), 4.08-3.96 (m, 2H), 3.92-3.82 (m, 1H), 3.87 (s, 3H), 3.36-3.23 (m, 1H), 3.08-2.96 (m, 1H), 2.14-2.23 (m, 2H), 1.94-1.42 (m, 7H), 0.96-0.86 (m, 3H), 0.83-0.71 (m, 3H); Calculated for C₂₄H₂₉NO₇, 443.19; observed (M+H)⁺ 444.8.

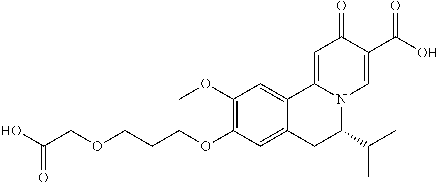 [SMILES: COc1cc2c(cc1OCCCOCC(=O)O)C[C@@H](C(C)C)n1cc(C(=O)O)c(=O)cc1-2]

### Example 5

Synthesis of (S)-9-(3-(carboxymethoxy)propoxy)-6-isopropyl-10-methoxy-2-oxo-6,7-dihydro-2H-pyrido[2,1-a]isoquinoline-3-carboxylic acid (65025-C—OH): (S)-9-(3-(carboxymethoxy)propoxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with methyl 2-(3-bromopropoxy)acetate. ¹H NMR (300 MHz, CDCl₃): δ (ppm) 8.58 (s, 1H), 7.38 (s, 1H), 7.24 (s, 1H), 6.80 (s, 1H), 4.24 (t, J=6.0 Hz, 2H), 4.16 (s, 2H), 4.00-3.94 (m, 1H), 3.94 (s, 3H), 3.81 (t, J=6.0 Hz, 2H), 3.42-3.31 (m, 1H), 3.15-3.05 (m, 1H), 2.26-2.15 (m, 2H), 1.93-1.78 (m, 1H), 0.96 (d, J=6.6 Hz, 3H), 0.82 (d, J=6.9 Hz, 3H); Calculated for C₂₃H₂₇NO₈, 445.17; observed (M+H)⁺ 446.7.

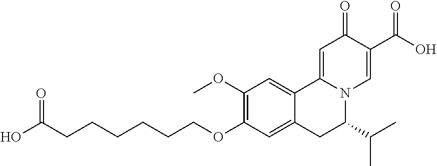 [SMILES: COc1cc2c(cc1OCCCCCCC(=O)O)C[C@@H](C(C)C)n1cc(C(=O)O)c(=O)cc1-2]

### Example 6

Synthesis of (S)-9-((6-carboxyhexyl)oxy)-6-isopropyl-10-methoxy-2-oxo-6,7-dihydro-2H-pyrido[2,1-a]isoquinoline-3-carboxylic acid (65042-E-OH): (S)-9-((6-carboxyhexyl)oxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with methyl 7-bromoheptanoate. ¹H NMR (300 MHz, CDCl₃): δ (ppm) 8.43 (s, 1H), 7.16 (s, 1H), 7.03 (s, 1H), 6.70 (s, 1H), 4.05-3.99 (m, 2H), 3.94-3.80 (m, 1H), 3.89 (s, 3H), 3.36-3.24 (m, 2H), 2.31-2.22 (m, 2H), 1.90-1.32 (m, 10H), 0.90 (d, J=6.3 Hz, 3H), 0.77 (d, J=6.3 Hz, 3H); Calculated for C₂₅H₃₁NO₇, 457.21; observed (M+H)⁺ 458.8.

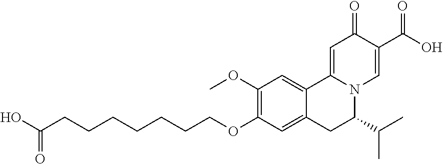 [SMILES: COc1cc2c(cc1OCCCCCCCC(=O)O)C[C@@H](C(C)C)n1cc(C(=O)O)c(=O)cc1-2]

### Example 7

Synthesis of (S)-9-((7-carboxyheptyl)oxy)-6-isopropyl-10-methoxy-2-oxo-6,7-dihydro-2H-pyrido[2,1-a]isoquinoline-3-carboxylic acid (65063-A-OH): (S)-9-((7-carboxyheptyl)oxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with ethyl 8-bromooctanoate. ¹H NMR (300 MHz, CDCl₃): δ (ppm) 8.54 (s, 1H), 7.33 (s, 1H), 7.21 (s, 1H), 6.73 (s, 1H), 4.10-4.03 (m, 2H), 3.94 (s, 3H), 3.93-3.86 (m, 1H), 3.41-3.31 (m, 1H), 3.11-3.04 (m, 1H), 2.37 (t, J=7.2 Hz, 2H), 1.94-1.75 (m, 3H), 1.75-1.58 (m, 2H), 1.58-1.32 (m, 6H), 0.95 (d, J=6.6 Hz, 3H), 0.82 (d, J=6.9 Hz, 3H); Calculated for C₂₆H₃₃NO₇, 471.23; observed (M+H)⁺ 472.7.

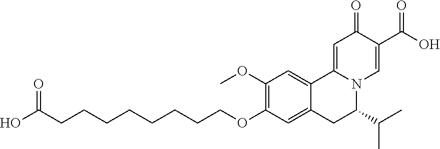 [SMILES: COc1cc2c(cc1OCCCCCCCCC(=O)O)C[C@@H](C(C)C)n1cc(C(=O)O)c(=O)cc1-2]

### Example 8

Synthesis of (S)-9-((8-carboxyoctyl)oxy)-6-isopropyl-10-methoxy-2-oxo-6,7-dihydro-2H-pyrido[2,1-a]isoquinoline-3-carboxylic acid (65075): (S)-9-((8-carboxyoctyl)oxy)-6-isopropyl-10-methoxy-2-oxo-6,7-dihydro-2H-pyrido[2,1-a]isoquinoline-3-carboxylic acid was prepared according to the procedure of example 1, except that (S)-ethyl 9-hydroxy-6-isopropyl-10-methoxy-2-oxo-6,7-dihydro-2H-pyrido[2,1-a]isoquinoline-3-carboxylate was replaced with (S)-ethyl 9-hydroxy-6-isopropyl-10-methoxy-2-oxo-6,7-dihydro-2H-pyrido[2,1-a]isoquinoline-3-carboxylate and methyl 2-bromoacetate was replaced with ethyl 9-bromononanoate. ¹H NMR (300 MHz, CDCl₃): δ (ppm) 8.52 (s, 1H), 7.29 (s, 1H), 7.21 (s, 1H), 6.73 (s, 1H), 4.12-4.03 (m, 2H), 3.94 (s, 3H), 3.92-3.86 (m, 1H), 3.40-3.30 (m, 1H), 3.11-3.04 (m, 1H), 2.36 (t, J=7.2 Hz, 2H), 1.94-1.76 (m, 3H), 1.70-1.55 (m, 2H), 1.55-1.30 (m, 8H), 0.95 (d, J=6.6 Hz, 3H), 0.82 (d, J=6.9 Hz, 3H); Calculated for C₂₇H₃₅NO₇, 485.24; observed (M+H)⁺ 486.8.

### Formulations

The present invention also relates to compositions or formulations which comprise the hepatoselective Polyadenylating Polymerases 5 and 7 (PAPD 5 & 7) inhibitors according to the present invention. In general, the compositions of the present invention comprise an effective amount of one or more of the compounds of the disclosure and salts thereof according to the present invention which are effective for treating or preventing diseases that involve Polyadenylating Polymerases 5 and 7 activity or that are caused by infectious agents that require these Polyadenylating Polymerases for their replication such as hepatitis A virus.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known hepatitis therapies and cancer therapies. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more compounds of the disclosure according to the present invention and one or more excipients; from about 0.01 mg to about 100 mg of one or more compounds of the disclosure according to the present invention and one or more excipients; and from about 0.1 mg to about 10 mg of one or more compounds of the disclosure according to the present invention; and one or more excipients.

### Procedures

The following procedures can be utilized in evaluating and selecting compounds as hepatoselective Polyadenylating Polymerases 5 and 7 (PAPD 5 & 7) inhibitors.

PAPD 5/7 Biochemical assay: PAPD5 (accession #: NM_001040285) and PAPD7 (accession #: NM_0069999) open reading frames were recombined into Flag-tagged pCDNA3.1 vectors to make plasmids pCMV-FlagD5 and pCMV-FlagD7, respectively. HEK293 cells grown in 10 cm dishes were then transiently transfected with 5 ug pCMV-FlagD5 or pCMV-FlagD7. Two days post the transfection, cell lysates were prepared (Cell Signaling Cat #: 9803S) and tagged PAPD5 and PAPD7 polypeptides were precipitated with M2 anti-Flag antibody (Sigma Cat #: A2220). PAPD5 or PAP7 bound on agarose beads were directly utilized for polyadenylating reaction in 10 ul buffer containing 10 mM Tris-HCl (pH 8.0), 100 mM KCl, 3.2 mM MgCl₂, 1 mM ATP and 100 nM P32-labeled RNA oligonucleotides (5′-GCCUUUCAUCUCUAACUGCGAAAAAAAAAA-3′). The polyadenylating reactions of RNA oligonucleotides were carried out at 37° C. for 20 minutes and 3 hours for the PAPD5 and PAPD7 polypeptides, respectively. The reaction was terminated by the addition of an equal volume of 2×RNA loading buffer (NEB, Cat #: B0363S). A 2.5 ul fraction of tail-elongated product was resolved in 15% Urea-Tris-Borate-EDTA (Urea-TBE) gel (Themofisher Cat #: EC6865BOX), and the radioactive signals were scanned with a Typhoon Phosphorimager.

To evaluate the inhibitory activity of compounds of the disclosure against PAPD5/7's enzymatic function, compounds of the disclosure were dissolved in DMSO at 8 serially diluted concentrations and add 1 ul of solution of compounds of the disclosure into 8 ul polyadenylating reaction buffer with final concentrations ranging from 1 uM to 0.4 nM. Addition of compound into the reaction buffer took place in prior to the addition of 1 ul of P³²-labeled RNA oligos. Inhibitory potency was measured based on the length of poly(A) tail and compared with that of RG7834 at the identical concentration.

### Assay for Antiviral Activity Against Human Hepatitis A Virus (Hepatovirus A).

Assays for the anti-HAV activity of the hepatoselective DHQ compounds were carried out in Huh-7 human hepatoma cells using the 18f-NLuc reporter virus, which expresses nanoluciferase (NLuc) from sequence inserted into the polyprotein coding region of the HM175/18f HAV genome between the VP1pX and 2B coding sequences (Yamane et al., 2019). The method was modified minimally from that described by Misumi et al. for iminosugar compounds (Misumi et al., 2021). Cells (approximately 104) were plated in wells of a 96-well plate with Dulbecco's modified Eagle medium (DMEM) and 10% fetal bovine serum. The cells were inoculated 24 hrs later with 18f-NLuc virus (approximately 10⁶-10⁷ genome equivalents) in medium containing increasing concentrations of the compounds of interest. The cells were re-incubated for 48 hrs at 37° C. in a 5% CO₂ environment, then lysed in 1× passive lysis buffer (Promega) for 15 min at room temperature. Cell lysates were transferred to an opaque white 96-well plate (Corning, 3912) and NLuc assay carried out using the NLuc GLOW Assay kit (Nanolight Technology). Luminescence was measured using a Biotek Synergy II multi-mode plate reader (BioTek Instruments). The percent inhibition of replication was calculated by determining the reduction in NLuc activity from that expressed by infected cells incubated in parallel in the absence of compound, and 50% inhibitory endpoints estimated by fitting the data to a four parameter variable slope logistic curve using Prism 9 for Mac OS software (GraphPad). Cell toxicity of the compounds was assessed using the WST-1 Cell Proliferation Reagent (Sigma-Aldrich) at the end of the treatment period in parallel cultures of uninfected cells, with WST-1 reagent added to cultures for 1 hr prior to harvest and O.D. reading.

Compounds of the disclosure can also be assessed to determine their utility for as PAPD 5 and 7 inhibitors using the methods described in WO2017016960, WO2019123285, WO2020106816A1, WO2018022282, WO2017216685, and WO2018130152 which are incorporated herein by reference.

Exemplary results for compounds of the disclosure are shown in table 6.

TABLE 6
Exemplary in vitro screening data for compounds of the disclosure

| Compound | EC₅₀ (μM) | CC₅₀ (μM) |
|---|---|---|
| Example 6 (65042-E-OH) | 0.278 | >10 |
| Example 8 (65075) | 0.0182 | >10 |

Protocol for pharmacokinetic and tissue distribution studies. The pharmacokinetics of the compounds was studied in CD1 mice after intravenous (IV) and oral gavage (PO) administration. Mouse PK studies was conducted at 3 mg/kg (IV) and 8 mg/kg (PO). Plasma samples obtained from dosed animals, at ten time points, including 5, 15 and 30 minutes and 1, 1.5, 2, 4, 6, 8 and 24 hr post-dose for processing to plasma, were prepared for analysis by means of a single step protein precipitation technique, by adding 200 μL of acetonitrile containing an IS mixture (labetalol, imipramine and diclofenac) to 50 μL aliquots of individual subject samples. Samples were mixed by vortex for homogeneity and then subjected to centrifugation at 4000 rpm for 10 min. The supernatant (200 μL) was collected and injected into the LC-MS/MS for analysis. Pharmacokinetic parameters will be calculated using established non-compartmental methods. The liver distribution of the compounds was studied in the PO group and liver tissues were collected at each time point after PO administration. Accurately weighed whole liver tissues were collected in a 50 mL tube, and 3 volumes per weight of water will be added. Tissue samples were homogenized and 50 μL of the homogenates will be processed along with plasma samples as described above.

Exemplary result for PK studies for compounds of the disclosure are shown in tables 7 through 11. Table 7 and 8 provide pharmacokinetic data and calculated PK properties after a 3 mg/kg IV administration of 65042-E-OH (Example 6) to CD1 mice. Tables 9 and 10 provide pharmacokinetic data and calculated PK properties after an 8 mg/kg oral administration of 65042-E-OH (Example 6) to CD1 mice. Table 11 provides a comparison of plasma concentration to liver concentration in CD1 mice after oral administration of 8 mg/kg of 65042-E-OH (Example 6) to CD1 mice. The results in 7 through 11 demonstrate the surprising and unexpected result that compounds of the disclosure are taken up by the liver, a feature not observed in previous generations of PAPD 5 and 7 inhibitors.

TABLE 7
PK data for 3 mg/kg IV dose of 65042-
E-OH (Example 6) in CD1 mice

| Time | Concentration (ng/mL) | | | Mean | SD | CV |
|---|---|---|---|---|---|---|
| (h) | Mouse 1 | Mouse 2 | Mouse 3 | (ng/mL) | (ng/mL) | (%) |
| 0.0833 | 817 | 979 | 689 | 828 | 145 | 17.5 |
| 0.25 | 82.3 | 103 | 73.0 | 86 | 15 | 17.8 |
| 0.5 | 32.8 | 31.7 | 24.9 | 29.8 | 4.3 | 14.4 |
| 1 | 11.4 | 8.52 | 6.79 | 8.9 | 2.3 | 26.2 |
| 2 | 5.61 | 5.51 | 2.14 | 4.42 | 1.98 | 44.7 |
| 4 | 1.85 | 1.69 | BLOQ | 1.77 | NA | NA |
| 8 | 1.08 | BLOQ | BLOQ | NA | NA | NA |
| 24 | BLOQ | BLOQ | BLOQ | NA | NA | NA |

*BLOQ = Below the lower level of quantitation.

TABLE 8
Calculated PK parameters of 3 mg/kg IV dose of 65042-E-OH (Example 6) in CD1 mice

| PK parameters | Unit | Mouse 1 | Mouse 2 | Mouse 3 | Mean | SD | CV(%) |
|---|---|---|---|---|---|---|---|
| Cl_obs | mL/min/kg | 187 | 166 | 242 | 198 | 39 | 19.8 |
| T_{1/2} | h | 2.72 | 1.27 | 0.442 | 1.48 | 1.15 | 78.1 |
| C₀ | ng/mL | 2572 | 3016 | 2115 | 2568 | 450 | 17.5 |
| AUCₗₐₛₜ | h*ng/mL | 263 | 298 | 205 | 255 | 47 | 18.4 |
| AUC_{Inf} | h*ng/mL | 268 | 301 | 206 | 258 | 48 | 18.6 |
| AUC_{_{—}}_{% Extrap —}obs | % | 1.59 | 1.03 | 0.662 | 1.09 | 0.46 | 42.6 |
| MRT_{Inf —}obs | h | 0.502 | 0.230 | 0.128 | 0.287 | 0.193 | 67.4 |
| AUCₗₐₛₜ/D | h*mg/mL | 87.8 | 99.2 | 68.3 | 85.1 | 15.6 | 18.4 |
| V_{ss —}obs | L/kg | 5.63 | 2.29 | 1.87 | 3.26 | 2.06 | 63.1 |

TABLE 9
PK data for 8 mg/kg oral dose of 65042-
E-OH (Example 6) in CD1 mice.

| Time | Concentration (ng/mL) | | | Mean | SD | CV |
|---|---|---|---|---|---|---|
| (h) | Group 1 | Group 2 | Group 3 | (ng/mL) | (ng/mL) | (%) |
| 0.25 | 18.4 | 10.3 | 19.5 | 16.1 | 5.0 | 31.3 |
| 0.5 | 9.40 | 9.93 | 9.00 | 9.44 | 0.47 | 4.94 |
| 1 | 4.10 | 5.94 | 9.27 | 6.44 | 2.62 | 40.7 |
| 2 | 5.41 | 4.55 | 2.29 | 4.08 | 1.61 | 39.5 |
| 4 | 3.68 | 3.03 | 4.12 | 3.61 | 0.55 | 15.2 |
| 6 | 2.85 | 1.53 | 2.42 | 2.27 | 0.67 | 29.7 |
| 8 | 6.65 | 2.11 | BLOQ | 4.38 | NA | NA |
| 24 | BLOQ | BLOQ | BLOQ | NA | NA | NA |

*BLOQ = Below the lower level of quantitation.

TABLE 10
Calculated PK parameters of 8 mg/kg oral dose
of 65042-E-OH (Example 6) in CD1 mice

| PK parameters | Unit | Mean |
|---|---|---|
| T_{1/2} | h | NA |
| Tₘₐₓ | h | 0.250 |
| Cₘₐₓ | ng/mL | 16.1 |
| AUCₗₐₛₜ | h*ng/mL | 34.7 |
| AUC_{Inf} | h*ng/mL | NA |
| AUC_{_{—}}_{% Extrap}_{_{—}}obs | % | NA |
| MRT_{Inf}_{_{—}}obs | h | NA |
| AUCₗₐₛₜ/D | h*mg/mL | 4.33 |
| F | % | 5.09 |

TABLE 11
Comparison of liver and plasma concentration
of 65042-E-OH (Example 6) in CD1 mice after
8 mg/kg oral dose of 65042-E-OH (Example 6)

| | | | | Ratio | | | |
|---|---|---|---|---|---|---|---|
| Time | Animal | Liver | Plasma | (liver/ | | | |
| (h) | ID | (ng/g) | (ng/mL) | plasma) | Mean | SD | CV |
| 0.25 | M4 | 199 | 18.4 | 10.8 | 23.8 | 15.9 | 66.7 |
| | M5 | 428 | 10.3 | 41.6 | | | |
| | M6 | 372 | 19.5 | 19.1 | | | |
| 0.5 | M7 | 366 | 9.40 | 38.9 | 32.9 | 7.7 | 23.3 |
| | M8 | 241 | 9.93 | 24.3 | | | |
| | M9 | 321 | 9.00 | 35.6 | | | |
| 1 | M10 | 259 | 4.10 | 63.1 | 48.9 | 12.5 | 25.6 |
| | M11 | 237 | 5.94 | 39.9 | | | |
| | M12 | 404 | 9.27 | 43.6 | | | |
| 2 | M13 | 377 | 5.41 | 69.6 | 60.9 | 11.9 | 19.6 |
| | M14 | 299 | 4.55 | 65.8 | | | |
| | M15 | 108 | 2.29 | 47.3 | | | |
| 4 | M16 | 106 | 3.68 | 28.9 | 38.2 | 8.1 | 21.1 |
| | M17 | 128 | 3.03 | 42.2 | | | |
| | M18 | 179 | 4.12 | 43.5 | | | |
| 6 | M19 | 130 | 2.85 | 45.6 | 32.3 | 11.8 | 36.7 |
| | M20 | 43.2 | 1.53 | 28.2 | | | |
| | M21 | 55.6 | 2.42 | 23.0 | | | |
| 8 | M22 | 106 | 6.65 | 15.9 | 16.8 | NA | NA |
| | M23 | 37.6 | 2.11 | 17.8 | | | |
| | M24 | 45.6 | BLOQ | NA | | | |
| 24 | M25 | NA | BLOQ | NA | NA | NA | NA |
| | M26 | NA | BLOQ | NA | | | |
| | M27 | NA | BLOQ | NA | | | |

Protocol for identification of compounds that are substrates of transporters OATPIBI and OATPIB3. Transfected HEK293 and vector control HEK293 cells were cultured in 24-well poly-D-lysine or collagen-coated tissue culture plates (as appropriate) and treated with a test compound at two concentrations (0.5 and 5 μM) for 2 and 10 minutes respectively in transport buffer (HBSSg, pH 7.4, pre-warmed to 37° C.). At a defined time point, the uptake experiments were stopped by removing dosing solution followed by two washes of ice-cold HBSSg buffer. The cells were lysed for 2 minutes with 75% ACN containing an appropriate internal standard (when available). Lysis buffer were chilled to −20° C., and the plate was kept on ice during lysis. The cell lysate was transferred into a 96-well plate for sample analysis with LC-MS/MS analysis to determine concentration of test compound in cell lysate. The uptake of test compounds was normalized by total cell protein using the BCA Protein Assay Kit following the manufacturer's protocol. The inhibitor Rifamycin SV was used to block OATP1B1 and OATP1B3 transporter activity in order to determine uptake ratios.

Results of OATP1B1 and OATP1B3 transporter studies for exemplary compounds of the disclosure, DHQ-1, and the positive control Estradiol 17-3 Glucuronide are shown in table 12 and table 13. Data for compounds of the disclosure and the positive control tested using Mock cells (HEK293 cells without transfection of OATP1B1 and OATP1B3 transporter) are also shown in tables 12 and 13. The results in tables 12 and 13 demonstrate the surprising and unexpected result that compounds of the disclosure are substrates for the OATP1B1 and OATP1B3 transporters, a feature not observed in previous generations of PAPD 5 and 7 inhibitors.

TABLE 12
Data from OATP1B1 transporter activity of compounds
of the disclosure using in OATP1B1-HEK293 Cells

| | Test | | Uptake Ratio |
|---|---|---|---|
| | Concentration | Uptake Ratio | (Transporter_{(-Inhibitor)}/ |
| Compound | (μM) | (−/+Inhibitor) | Mock_{(-Inhibitor)}) |
| Estradiol 17-β | 10 | 81.84 | 48.70 |
| Glucuronide | | | |
| Example 6 | 1 | 11.17 | 18.99 |
| (65042-E-OH) | | | |
| DHQ-1 | 1 | 0.38 | 0.71 |

Mock cells are normal HEK293

TABLE 13
Data from OATP1B3 transporter activity of compounds
of the disclosure using in OATP1B3-HEK293 Cells

| | Test | | Uptake Ratio |
|---|---|---|---|
| | Concentration | Uptake Ratio | (Transporter_{(-Inhibitor)}/ |
| Compound | (μM) | (−/+Inhibitor) | Mock_{(-Inhibitor)}) |
| Estradiol 17-β | 10 | 86.15 | 98.86 |
| Glucuronide | | | |
| Example 6 | 1 | 11.57 | 24.80 |
| (65042-E-OH) | | | |
| DHQ-1 | 1 | 0.42 | 0.56 |

Additional exemplary compounds of the present invention are evaluated for anti-HAV activity in accordance with certain assays described hereinabove. As discussed above, assays for the anti-HAV activity of the hepatoselective DHQ compounds were carried out in Huh-7 human hepatoma cells using the 18f-NLuc reporter virus, which expresses nanoluciferase (NLuc) from sequence inserted into the polyprotein coding region of the HM175/18f HAV genome between the VP1pX and 2B coding sequences (Yamane et al., 2019). The method was modified minimally from that described by Misumi et al. for iminosugar compounds (Misumi et al., 2021). Cells (approximately 104) were plated in wells of a 96-well plate with Dulbecco's modified Eagle medium (DMEM) and 10% fetal bovine serum. The cells were inoculated 24 hrs later with 18f-NLuc virus (approximately 10⁶-107 genome equivalents) in medium containing increasing concentrations of the compounds of interest. The cells were re-incubated for 48 hrs at 37° C. in a 5% CO₂ environment, then lysed in 1× passive lysis buffer (Promega) for 15 min at room temperature. Cell lysates were transferred to an opaque white 96-well plate (Corning, 3912) and NLuc assay carried out using the NLuc GLOW Assay kit (Nanolight Technology). Luminescence was measured using a Biotek Synergy II multi-mode plate reader (BioTek Instruments). The percent inhibition of replication was calculated by determining the reduction in NLuc activity from that expressed by infected cells incubated in parallel in the absence of compound, and 50% inhibitory endpoints estimated by fitting the data to a four parameter variable slope logistic curve using Prism 9 for Mac OS software (GraphPad). Cell toxicity of the compounds was assessed using the WST-1 Cell Proliferation Reagent (Sigma-Aldrich) at the end of the treatment period in parallel cultures of uninfected cells, with WST-1 reagent added to cultures for 1 hr prior to harvest and O.D. reading.

The results for certain exemplary compounds of the present invention are shown in Table 14 (below).

TABLE 14

| | | EC₅₀ |
|---|---|---|
| | | HAV |
| Compound | Structure | (nM) |
| Example A | 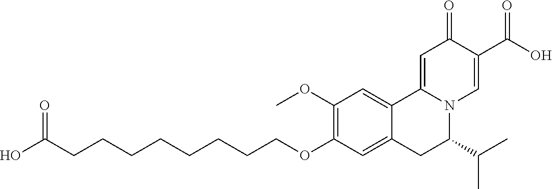 [SMILES: COc1cc2c(cc1OCCCCCCCCC(=O)O)C[C@@H](C(C)C)n1cc(C(=O)O)c(=O)cc1-2] | 67 |
| | Chemical Formula: C₂₇H₃₅NO₇ | |
| | Exact Mass: 485.24 | |
| | Molecular Weight: 485.57 | |
| Example B | 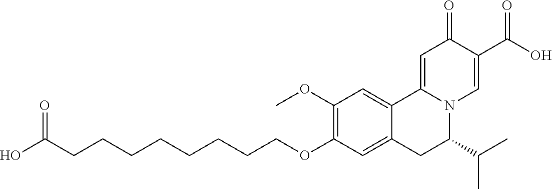 [SMILES: COc1cc2c(cc1OCCCCCCCCC(=O)O)C[C@@H](C(C)C)n1cc(C(=O)O)c(=O)cc1-2] | 63 |
| | Chemical Formula: C₂₉H₃₉NO₇ | |
| | Exact Mass: 513.27 | |
| | Molecular Weight: 513.62 | |
| Example C | 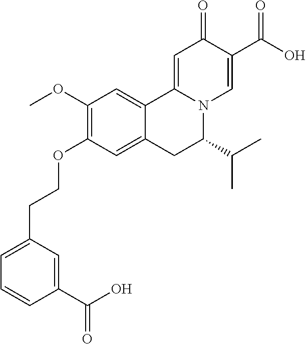 [SMILES: COc1cc2c(cc1OCCc1cccc(C(=O)O)c1)C[C@@H](C(C)C)n1cc(C(=O)O)c(=O)cc1-2] | 325 |
| | Chemical Formula: C₂₇H₂₇NO₇ | |
| | Exact Mass: 477.18 | |
| | Molecular Weight: 477.51 | |
| Example D | 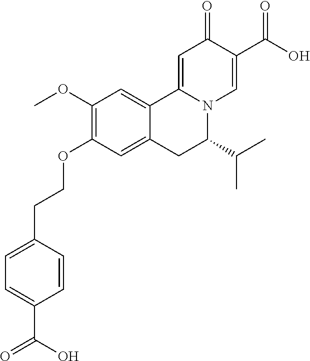 [SMILES: COc1cc2c(cc1OCCc1ccc(C(=O)O)cc1)C[C@@H](C(C)C)n1cc(C(=O)O)c(=O)cc1-2] | 245 |
| | Chemical Formula: C₂₇H₂₇NO₇ | |
| | Exact Mass: 477.18 | |
| | Molecular Weight: 477.51 | |
| Example E | 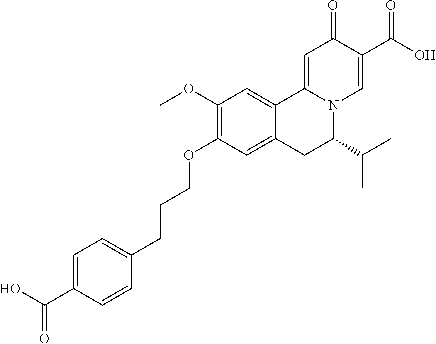 [SMILES: COc1cc2c(cc1OCCCc1ccc(C(=O)O)cc1)C[C@@H](C(C)C)n1cc(C(=O)O)c(=O)cc1-2] | 382 |
| | Chemical Formula: C₂₈H₂₉NO₇ | |
| | Exact Mass: 491.19 | |
| | Molecular Weight: 491.53 | |
| Example F | 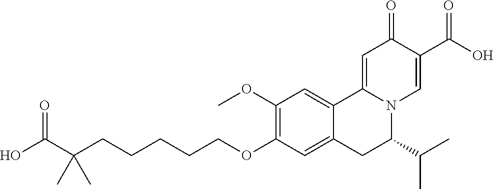 [SMILES: COc1cc2c(cc1OCCCCCC(C)(C)C(=O)O)C[C@@H](C(C)C)n1cc(C(=O)O)c(=O)cc1-2] | 25 |
| | Chemical Formula: C₂₇H₃₅NO₇ | |
| | Exact Mass: 485.24 | |
| | Molecular Weight: 485.58 | |
| Example G | 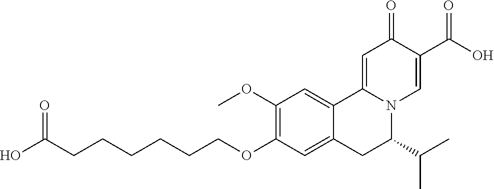 [SMILES: COc1cc2c(cc1OCCCCCCC(=O)O)C[C@@H](C(C)C)n1cc(C(=O)O)c(=O)cc1-2] | 278 |
| Example H | 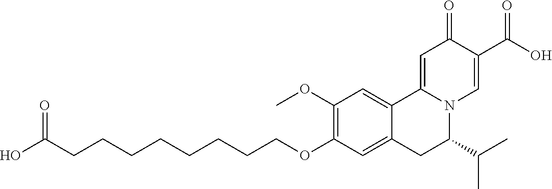 [SMILES: COc1cc2c(cc1OCCCCCCCCC(=O)O)C[C@@H](C(C)C)n1cc(C(=O)O)c(=O)cc1-2] | 18.2 |

1. A method for:
treating disease due to infection with Hepatitis A Virus (HAV); or
preventing HAV-related disease in a subject known or suspected to have been exposed to the virus; said method comprising administering to a subject in need thereof, an effective amount of a compound having formula (I): 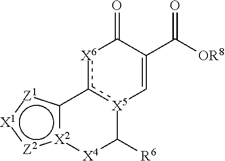
including hydrates, solvates, pharmaceutically acceptable salts, prodrugs and complexes thereof, wherein:
X¹ is selected from the group consisting of 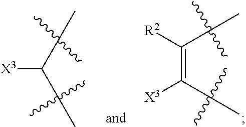
When X¹ is 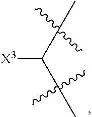
X² is nitrogen;
When X¹ is 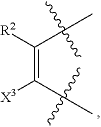
X² is carbon;
Z¹ is selected from the group consisting of N and CR¹;
Z² is selected from the group consisting of N and CR⁴;
Z¹ and Z² are not N at the same time;
X³ is selected from the group consisting of CH₂R, —OR, —NR₃R and 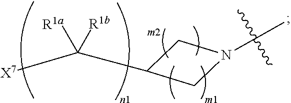
X⁴ is selected from the group consisting of CHR⁵, O, S, SO, and SO₂;
X⁵ is selected from the group consisting of N and C;
X⁶ is selected from the group consisting of CR⁷ and NR⁷;
When X⁵ is N, X⁶ us CR⁷;
When X⁵ is C, X⁶ is NR⁷;
When X⁵ is N, X⁴ is CHR⁵(CH₂)ₚ, p is 0, 1, or 2;
When X⁵ is N, X⁴ is O(CH₂)ₚ, S(CH₂)ₚ, SO(CH₂)ₚ, and SO₂(CH₂)ₚ, p is 0, 1, or 2;
When X⁵ is C, X⁴ is selected from the group consisting of O(CH₂)ₚ, S(CH₂)ₚ, SO(CH₂)ₚ, and SO₂(CH₂)ₚ, p is 0, 1, or 2;
R is selected from the group consisting of 1 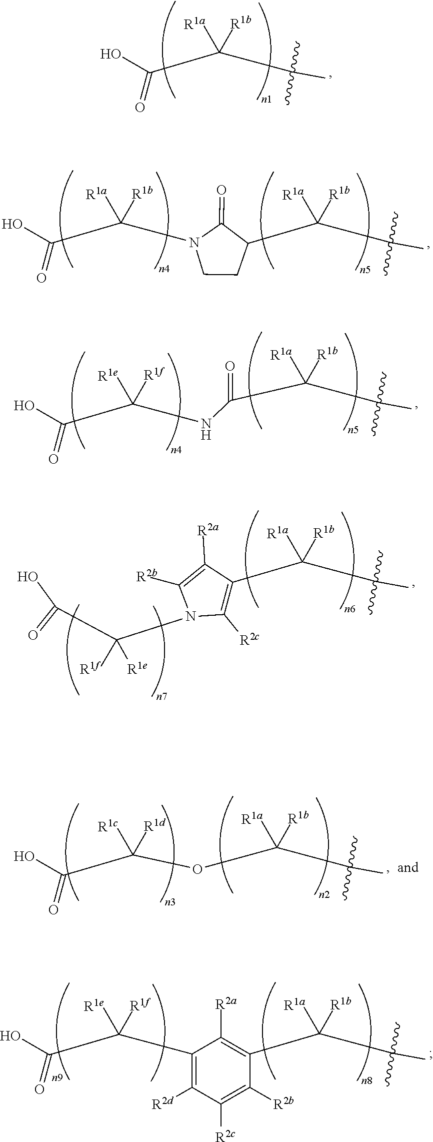
n¹ is 1, 2, 3, 4, 5, 6, 7, 8, 9, 10, 11, 12, 13, 14, 15, or 16;
n² is 2, 3, 4, 5, 6, 7, or 8;
n³ is 1, 2, 3, 4, 5, 6, 7, or 8;
n⁴ is 1, 2, 3, 4, 5, 6, or 7;
n⁵ is 1, 2, 3, 4, 5, 6, or 7;
n⁶ is 1, 2, 3, 4, 5, 6, or 7;
n⁷ is 1, 2, 3, 4, 5, 6, or 7;
n⁸ is 1, 2, 3, 4, 5, 6, or 7;
n⁹ is 0, 1, 2, 3, 4, 5, 6, or 7;
The sum of n² and n³ does not exceed 14:
The sum of n⁴ and n⁵ does not exceed 14:
The sum of n⁶ and n⁷ does not exceed 14:
The sum of n⁸ and n⁹ does not exceed 14:
m¹ is 1, 2, or 3;
m² is 0, 1, 2, or 3;
R¹ is selected from the group consisting of H, halogen, OH, CN, C₁₋₆ alkyl, C₁₋₆ haloalkyl, C₃₋₇ branched alkyl, and C₃₋₇ cycloalkyl;
R^{1a} is at each occurrence independently selected from the group hydrogen, halogen, C₁₋₆ alkyl, C₃₋₇ branched alkyl, and C₃₋₇ cycloalkyl;
R^{1b} is at each occurrence independently selected from the group hydrogen, halogen, C₁₋₆ alkyl, C₃₋₇ branched alkyl, and C₃₋₇ cycloalkyl;
R^{1c} is at each occurrence independently selected from the group hydrogen, halogen, C₁₋₆ alkyl, C₃₋₇ branched alkyl, and C₃₋₇ cycloalkyl;
R^{1d} is at each occurrence independently selected from the group hydrogen, halogen, C₁₋₆ alkyl, C₃₋₇ branched alkyl, and C₃₋₇ cycloalkyl;
R^{1e} is at each occurrence independently selected from the group hydrogen, halogen, C₁₋₆ alkyl, C₃₋₇ branched alkyl, and C₃₋₇ cycloalkyl;
R^{1f} is at each occurrence independently selected from the group hydrogen, halogen, C₁₋₆ alkyl, C₃₋₇ branched alkyl, and C₃₋₇ cycloalkyl;
R² is selected from the group consisting of H, halogen, C₁₋₆ alkyl, C₃₋₇ cycloalkyl, C₁₋₆ haloalkyl, C₃₋₇ branched alkyl, C₁₋₆ alkoxy, C₁₋₆ haloalkoxy, C₃₋₇ branched alkoxy, CN, and aromatic ring with 0-3 heteroatoms;
optionally, R¹ and R² are taken together to with the atoms to which they are bound to form a 5-7 membered ring that contains 0-2 heteroatoms;
R³ is selected from group consisting of hydrogen, C₁₋₆ alkyl, and C₂₋₆ alkene;
R⁴ is selected from the group consisting of H, halogen, C₁₋₆ alkyl, C₁₋₆ alkoxy, and CN;
or R³ and R⁴ are taken together with the atoms to which they are bound to form a 5-7 membered ring with 0-2 heteroatoms;
or R³ and R⁴ are taken together to with the atoms to which they are bound to form a 5-6 membered aromatic ring with 0-2 heteroatoms;
R⁵ is selected from the group consisting of H, C₁₋₆ alkyl, and C₃₋₇ branch alkyl;
R⁶ is selected from the group consisting of H, C₁₋₁₀ alkyl C₁₋₁₀ haloalkyl, C₃₋₇ branched alkyl, C₃₋₇ cycloalkyl, and optionally substituted aryl;
or R⁵ and R⁶ are taken together to with the atoms to which they are bound to form a 5-7 membered ring;
when X⁶ is CR⁷, R⁷ is selected from the group consisting of H, halogen, C₁₋₆ alkyl, C₃₋₇ cycloalkyl, C₃₋₇ branched alkyl, and CN;
when X⁶ is NR⁷, R⁷ is selected from the group consisting of H, C₁₋₆ alkyl, C₃₋₇ cycloalkyl, and C₃₋₇ branched alkyl; and
R⁸ is selected from the group consisting of is H and C₁₋₆ alkyl.

2. The method according to claim 1, wherein the compound of formula (I) is a compound of formula (II): 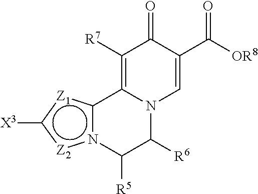
including hydrates, solvates, pharmaceutically acceptable salts, prodrugs, isotopic isomers, and complexes thereof.

3. The method according to claim 1, wherein the compound of formula (I) is a compound of formula (III) 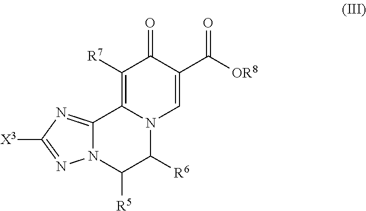
including hydrates, solvates, pharmaceutically acceptable salts, prodrugs, isotopic isomers, and complexes thereof.

4. The method according to claim 1, wherein the compound of formula (I) is a compound of formula (IV) 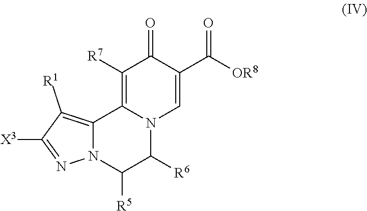
including hydrates, solvates, pharmaceutically acceptable salts, prodrugs, isotopic isomers, and complexes thereof.

5. The method according to claim 1, wherein the compound of formula (I) is a compound of formula (V) 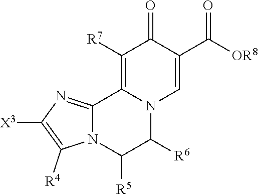
including hydrates, solvates, pharmaceutically acceptable salts, prodrugs, isotopic isomers, and complexes thereof.

6. The method according to claim 1, wherein the compound of formula (I) is a compound of formula (VI) 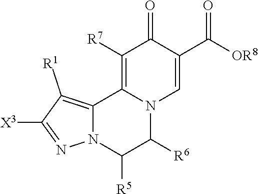
including hydrates, solvates, pharmaceutically acceptable salts, prodrugs, isotopic isomers, and complexes thereof.

7. The method according to claim 1, wherein the compound of formula (I), is a compound of formula (VII) 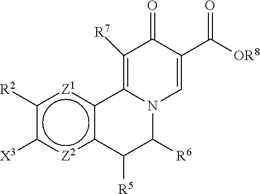
including hydrates, solvates, pharmaceutically acceptable salts, prodrugs, isotopic isomers, and complexes thereof.

8. The method according to claim 1, wherein the compound of formula (I) is a compound of formula (VIII) 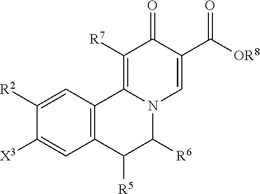
including hydrates, solvates, pharmaceutically acceptable salts, prodrugs, isotopic isomers, and complexes thereof.

9. The method according to claim 1, wherein the compound of formula (I) is a compound of formula (IX) 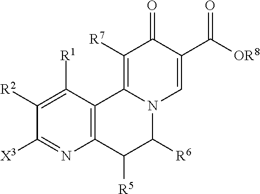
including hydrates, solvates, pharmaceutically acceptable salts, prodrugs, isotopic isomers, and complexes thereof.

10. The method according to claim 1, wherein the compound of formula (I) is a compound of formula (X) 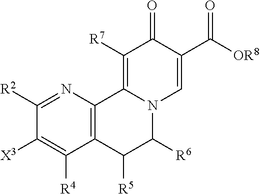
including hydrates, solvates, pharmaceutically acceptable salts, prodrugs, isotopic isomers, and complexes thereof.

11. The method according to claim 1, wherein the compound of formula (I) is a compound of formula (XI) 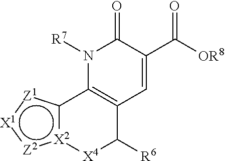
including hydrates, solvates, pharmaceutically acceptable salts, prodrugs, isotopic isomers, and complexes thereof.

12. The method according to claim 1, wherein the compound of formula (I) is a compound of formula (XII 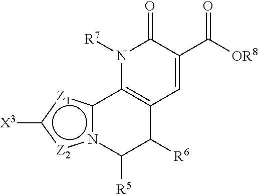
including hydrates, solvates, pharmaceutically acceptable salts, prodrugs, isotopic isomers, and complexes thereof.

13. The method according to claim 1, wherein the compound of formula (I) is a compound of formula (XIII) 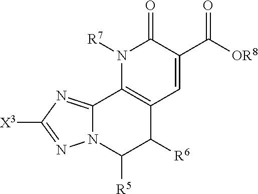
including hydrates, solvates, pharmaceutically acceptable salts, prodrugs, isotopic isomers, and complexes thereof.

14. The method according to claim 1, wherein the compound of formula (I) is a compound of formula (XIV) 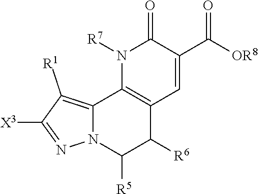
including hydrates, solvates, pharmaceutically acceptable salts, prodrugs, isotopic isomers, and complexes thereof.

15. The method according to claim 1, wherein the compound of formula (I) is a compound of formula (XV) 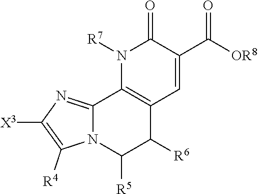
including hydrates, solvates, pharmaceutically acceptable salts, prodrugs, isotopic isomers, and complexes thereof.

16. The method according to claim 1, wherein the compound of formula (I) is a compound of formula (XVI) 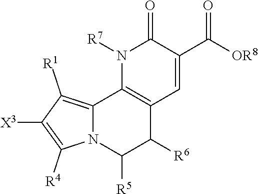
including hydrates, solvates, pharmaceutically acceptable salts, prodrugs, isotopic isomers, and complexes thereof.

17. The method according to claim 1, wherein the compound of formula (I) is a compound of formula (XVII) 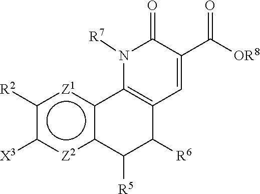
including hydrates, solvates, pharmaceutically acceptable salts, prodrugs, isotopic isomers, and complexes thereof.

18. The method according to claim 1, wherein the compound of formula (I) is a compound of formula (XVIII) 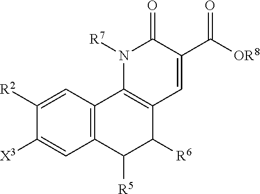
including hydrates, solvates, pharmaceutically acceptable salts, prodrugs, isotopic isomers, and complexes thereof.

19. The method according to claim 1, wherein the compound of formula (I) is a compound of formula (XIX) 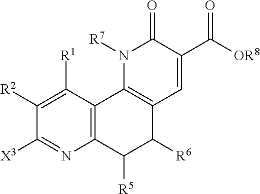
including hydrates, solvates, pharmaceutically acceptable salts, prodrugs, isotopic isomers, and complexes thereof.

20. The method according to claim 1, wherein the compound of formula (I) is a compound of formula (XX) 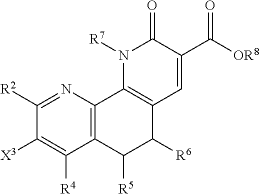
including hydrates, solvates, pharmaceutically acceptable salts, prodrugs, isotopic isomers, and complexes thereof.

21. The method according to claim 1, wherein the compound of formula (I) is a compound selected from:
(S)-9-(carboxymethoxy)-6-isopropyl-10-methoxy-2-oxo-6,7-dihydro-2H-pyrido [2,1-a]isoquinoline-3-carboxylic acid;
(S)-9-(3-carboxypropoxy)-6-isopropyl-10-methoxy-2-oxo-6,7-dihydro-2H-pyrido [2,1-a]isoquinoline-3-carboxylic acid;
(S)-9-(4-carboxybutoxy)-6-isopropyl-10-methoxy-2-oxo-6,7-dihydro-2H-pyrido[2,1-a]isoquinoline-3-carboxylic acid;
(S)-9-((5-carboxypentyl)oxy)-6-isopropyl-10-methoxy-2-oxo-6,7-dihydro-2H-pyrido[2,1-a]isoquinoline-3-carboxylic acid;
(S)-9-(3-(carboxymethoxy)propoxy)-6-isopropyl-10-methoxy-2-oxo-6,7-dihydro-2H-pyrido[2,1-a]isoquinoline-3-carboxylic acid;
(S)-9-((6-carboxyhexyl)oxy)-6-isopropyl-10-methoxy-2-oxo-6,7-dihydro-2H-pyrido[2,1-a]isoquinoline-3-carboxylic acid;
(S)-9-((7-carboxyheptyl)oxy)-6-isopropyl-10-methoxy-2-oxo-6,7-dihydro-2H-pyrido[2,1-a]isoquinoline-3-carboxylic acid;
(S)-9-((8-carboxyoctyl)oxy)-6-isopropyl-10-methoxy-2-oxo-6,7-dihydro-2H-pyrido[2,1-a]isoquinoline-3-carboxylic acid; and
and a hydrates, solvates, pharmaceutically acceptable salts, isotopic isomers, prodrugs and complexes thereof.

22. A method for treating disease due to infection with Hepatitis A Virus (HAV); or preventing HAV-related disease in a subject known or suspected to have been exposed to HAV, comprising administering an effective amount of a liver selective inhibitor of PAPD 5 and 7, to a subject in need thereof.

23. (canceled)
